(19) United States
(12) Patent Application Publication
(10) Pub. No.: US 2008/0161242 A1
(43) Pub. Date: Jul. 3, 2008

(54) High pressure treatment of proteins for reduced immunogenicity

(76) Inventors: Theodore W. Randolph, Niwot, CO (US); John F. Carpenter, Littleton, CO (US); Richard St. John, South San Francisco, CA (US); Lyndal K. Hesterberg, Boulder, CO (US); Matthew B. Seefeldt, Boulder, CO (US)

Correspondence Address: MORRISON & FOERSTER LLP, 755 PAGE MILL RD, PALO ALTO, CA, 94304-1018, US

(21) Appl. No.: 11/901,613

(22) Filed: Sep. 17, 2007

Related U.S. Application Data
(60) Provisional application No. 60/844,996, filed on Sep. 15, 2006.

(51) Int. Cl. A61K 38/00

(52) U.S. Cl. 514/12

(57) ABSTRACT

Protein compositions with reduced immunogenicity are disclosed, as well as methods for producing such compositions.

### CROSS REFERENCE TO RELATED APPLICATIONS

This non-provisional patent application claims priority under 35 USC §119(e) from U.S. Provisional Patent Application having Ser. No. 60/844,996, filed on Sep. 15, 2006, and titled HIGH PRESSURE TREATMENT OF PROTEINS FOR REDUCED IMMUNOGENICITY, wherein the entirety of said provisional patent application is incorporated herein by reference.

### FIELD OF THE INVENTION

This invention relates to methods for producing protein therapeutics having reduced immunogenicity by applying high pressure, and compositions containing such proteins. More particularly, the invention relates to recombinant proteins.

### BACKGROUND OF THE INVENTION

Therapeutic proteins provide enormous potential for the treatment of human disease. Dozens of protein therapeutics are currently available, with many more in clinical development. Unfortunately, protein aggregation is a common problem that arises during all phases of recombinant protein production, specifically during fermentation, purification, and long term storage (Schwarz, E., H. Lilie, et al. (1996), Biological Chemistry 377(7-8): 411-416; Carpenter, J. F., M. J. Pikal, et al. (1997), Pharmaceutical Research 14(8): 969-975; Baneyx, F. (1999), Current Opinion in Biotechnology 10(5): 411-421; Clark, E. D. (2001). Current Opinion in Biotechnology 12(2): 202-207; Chi, E. Y., S. Krishnan, et al. (2003), Protein Science 12(5): 903-913). Protein aggregation proceeds through specific pathways that are initiated by instability of the native protein conformation or colloid instability associated with protein-protein interactions. Conditions such as temperature, solution pH, ligands and cosolutes, salt type and concentration, preservatives, and surfactants all modulate protein structure and protein-protein interactions, and thus aggregation propensity. For aggregates that form from native protein instability, it appears that aggregates may form from protein structures present within the native state that demonstrate an expanded conformation and are often the result of non-specific hydrophobic interactions (Kendrick, B. S., J. F. Carpenter, et al. (1998), Proceedings of the National Academy of Sciences of the United States of America 95(24): 14142-14146; Kim, Y. S., J. S. Wall, et al. (2000), Journal of Biological Chemistry 275(3): 1570-1574). Consequently, aggregation can be controlled by the conformational stability of the native protein relative to that of the aggregation transition state. Recently, it has also been reported that proteins can form aggregates due to colloid instability, even in solution conditions which thermodynamically greatly favor the native conformation (Chi, E. Y., S. Krishnan, et al. (2003), Protein Science 12(5): 903-913). These molecular assembly reactions are a result of intermolecular attractions. For example, GCSF at pH 7.0 has been demonstrated to have a large ΔG_{unfolding}, yet the protein aggregates readily due to colloidal instability arising from attractive electrostatic interactions (Chi, E. Y., S. Krishnan, et al. (2003), Protein Science 12(5): 903-913). Due to myriad aggregation mechanisms in all proteins, it is not surprising that protein aggregation is a widespread problem in all aspects of protein processing, both in vivo and in vitro.

Soluble protein aggregates are often not recognized as “natural” by the immune system (possibly by exposure of a new epitope on the protein in the aggregate which is not exposed in the non-aggregated protein, or possibly by formation in the aggregate of a new, unrecognized epitope), with the result that the immune system is sensitized to the administered recombinant protein aggregate. In many instances, the immune system produces binding antibodies to the aggregates, which do not neutralize the therapeutic effect of the protein. However, in some cases, antibodies are produced that bind to the recombinant protein and interfere with the therapeutic activity thereby resulting in declining efficacy of the therapy. Furthermore, in some instances, repeated administration of a recombinant protein can cause acute and chronic immunologic reactions (see Schellekens, H., Nephrol. Dial. Transplant. 18:1257 (2003); Schellekens, H., Nephrol. Dial. Transplant. 20 [Suppl 6]:vi3-yl9 (2005); Purohit et al. J. Pharm. Sci. 95:358 (2006)).

During the development of the immune system, tolerance to an individual's own proteins develops, so that the immune system does not attack antigens normally present in the body (Singh et al., Nat. Clin. Pract. Rheumatol. 2:44 (2006)). This state of specific immunological tolerance to “self-components” involves both central and peripheral mechanisms. Central tolerance (negative selection) is a consequence of immature T cells receiving strong intracellular signaling while still resident in the thymus, resulting in clonal deletion of autoreactive cells. Peripheral tolerance occurs when the immune system becomes unreactive to an antigen present in the periphery, where, in contrast to the thymus, T cells are assumed to be functionally mature. Peripheral tolerance has been proposed to be the result of various mechanisms, including the development of antigen specific suppressor cells or other means of active tolerance, clonal deletion, and anergy. Autoreactive cells may be physically deleted by the induction of apoptosis after recognition of tolerizing antigen, may become anergic without deletion, or may be functionally inhibited by regulatory cytokines or cells.

Loss or “breaking” of tolerance can have serious effects including acute and chronic immune reactions and the development of autoimmune diseases. One devastating immune reaction can occur when upon repeated administration of a recombinant protein, tolerance is broken, and an immune response produced against the recombinant protein cross-reacts with the individual's endogenous protein. A mechanism for breaking self-tolerance was demonstrated in transgenic mice immune tolerant for human interferon-alpha 2. When preparations containing aggregates of recombinant human interferon-alpha 2b were administered to the mice, the mice lost tolerance for interferon-alpha 2 in a dose-dependent manner (see Hermeling et al., J Pharm Sci. 95:1084 (2006)).

A loss of tolerance to an endogenously produced protein has already been seen in patients using a preparation of recombinant erythropoietin. Certain preparations of erythropoietin sold under the trademark EPREX (Johnson & Johnson, New Brunswick, N.J.) in Europe were found to break the immune tolerance of patients for their own endogenous erythropoietin, leading to antibody-mediated pure red cell aplasia (PRCA). The exogenous erythropoietin preparation administered to correct a deficiency in red blood cell production elicited the patient's immune system to produce antibodies which neutralized endogenously produced erythropoietin causing a complete block in differentiation of red blood cells. The cause of the immune response has been attributed to leachates in the preparation which formed adjuvants with erythropoietin (Boven et al., Nephrol. Dial. Transplant. 20 Suppl 3:iii33 (2005)), although other factors, such as aggregates, may also be involved (Schellekens and Jiskoot, Nature Biotech. 24:613 (2006)).

A method for removal of soluble aggregates from protein therapeutics would thus contribute significantly to the safety of therapeutic proteins. One method of refolding proteins uses high pressure on solutions of proteins in order to disaggregate, unfold, and properly refold proteins. Such methods are described in U.S. Pat. No. 6,489,450, U.S. Patent Application Publication No. 2004/0038333, and International Patent Application WO 02/062827. Those disclosures indicated that certain high-pressure treatments of aggregated proteins or misfolded proteins resulted in recovery of disaggregated protein retaining biological activity (i.e., the protein was properly folded, as is required for biological activity) in good yields. U.S. Pat. No. 6,489,450, U.S. 2004/0038333, and WO 02/062827 are incorporated by reference herein in their entireties.

As illustrated below in the examples, however, conditions favorable to reduction or elimination of soluble aggregates in a protein preparation with high monomer content may not be similar to the conditions favorable to maximum yield of protein recovery from a highly aggregated solution. This distinction arises from the common observation that pressure treatment in many solution conditions can induce aggregation of monomeric species (Ferrao-Gonzales, A. D., S. O, Souto, et al. (2000), Proceedings of the National Academy of Sciences of the United States of America 97(12): 6445-6450, Kim, Y. S., T. W. Randolph, et al. (2002), Journal of Biological Chemistry 277(30): 27240-27246, Seefeldt, M. B., Y. S. Kim, et al. (2005), Protein Science 14(9): 2258-2266, Dzwolak, W. (2006), Biochimica Et Biophysica Acta-Proteins And Proteomics 1764(3): 470-480, Grudzielanek, S., V. Smirnovas, et al. (2006), Journal Of Molecular Biology 356(2): 497-509, Kim, Y. S., T. W. Randolph, et al. (2006), High-pressure studies on protein aggregates and amyloid fibrils. Amyloid, Prions, And Other Protein Aggregates, Pt C. 413: 237-253). Previous work with process-induced aggregates has resulted in the testing of solutions comprising of aggregates at a composition of >90% (Foguel, D., C. R. Robinson, et al. (1999), Biotechnology and Bioengineering 63(5): 552-558, Randolph, T. W., M. Seefeldt, et al. (2002), Biochimica Et Biophysica Acta-Protein Structure and Molecular Enzymology 1595(1-2): 224-234, Lefebvre, B. G., N. K. Comolli, et al. (2004), Protein Science 13(6): 1538-1546, Seefeldt, M. B. (2005), High pressure refolding of protein aggregates: Efficacy and thermodynamics, Doctoral thesis. Department of Chemical and Biological Engineering. Boulder, Colo., University of Colorado; Seefeldt, M. B., C. Crouch, et al. (2006), Journal of Biotechnology and Bioengineering In Press,). Consequently, high pressure refolding results have not been published for the aggregate dissociation of solutions comprising more monomeric material with less aggregate present. These solutions are more typical of the solutions that generate immunogenicity in patients. This difference is significant and imparts novelty since high pressure has been shown to induce aggregates for monomeric material (Ferrao-Gonzales, A. D., S. O, Souto, et al. (2000), Proceedings of the National Academy of Sciences of the United States of America 97(12): 6445-6450, Kim, Y. S., T. W. Randolph, et al. (2002), Journal of Biological Chemistry 277(30): 27240-27246, Seefeldt, M. B., Y. S. Kim, et al. (2005), Protein Science 14(9): 2258-2266, Dzwolak, W. (2006), Biochimica Et Biophysica Acta-Proteins And Proteomics 1764(3): 470-480, Grudzielanek, S., V. Smirnovas, et al. (2006), Journal Of Molecular Biology 356(2): 497-509, Kim, Y. S., T. W. Randolph, et al. (2006), High-pressure studies on protein aggregates and amyloid fibrils. Amyloid, Prions, And Other Protein Aggregates, Pt C. 413: 237-253).

As more recombinant human proteins become available on the market, the incidence of immunogenicity problems is rising. The antibodies formed against a therapeutic protein can result in serious clinical effects, such as loss of efficacy and neutralization of the endogenous protein with essential biological functions (Hermeling, S., D. J. A. Crommelin, et al. (2004), Pharmaceutical Research 21(6): 897-903). There are numerous factors which can result in the development of immunogenicity after treatment of therapeutic proteins, including amino acid sequence, glycosylation, chemical degradations, and physical degradation (Hermeling, S., D. J. A. Crommelin, et al. (2004), Pharmaceutical Research 21(6): 897-903). Immunogenicity related to amino acid sequence and glycosylation is species specific and can therefore be engineered away by ensuring that patients are dosed with human proteins using recombinant technology. Consequently, chemical and physical degradation remain the primary basis for the development of immunogenicity from protein therapeutics.

The amount of data on immunogenicity as a result of therapeutic protein administration is low, but the number of incidents are rising (Braun, A., L. Kwee, et al. (1997), Pharmaceutical Research 14: 1472-1478; Schellekens, H. (2002), Nature Reviews 1(6): 457-462; Schellekens, H. (2003), Nephrol Dial Transplant 18: 1257-1259; Deisenhammer, F., H. Schellekens, et al. (2004), J Neurol 251: 31-39; Hermeling, S., D. J. A. Crommelin, et al. (2004), Pharmaceutical Research 21(6): 897-903). A review of incidences of immune response occurring in patients after administration of protein therapeutics includes insulin, Factor VIII, epogen, growth hormone, interferon-alpha and interferon beta-1b (Moore, W. and P. Leppert (1980), Journal of Clinical Endocrinology and Metabolism 51: 691-697; Runkel, L., W. Meier, et al. (1998), Pharmaceutical Research 15(4): 641-649; Schellekens, H. (2003), Nephrol Dial Transplant 18: 1257-1259; Hermeling, S., D. J. A. Crommelin, et al. (2004), Pharmaceutical Research 21(6): 897-903; Hermeling, S., W. Jiskoot, et al. (2005), Pharmaceutical Research 22(6): 847-851; Hermeling, S., H. Schellekens, et al. (2006), Journal Of Pharmaceutical Sciences 95(5): 1084-1096). Immungenicity as a result of aggregate formation has been modeled further with studies of interferon alpha and beta-1b murine animal models as well as the examples set forth herein (Braun, A., L. Kwee, et al. (1997), Pharmaceutical Research 14: 1472-1478; Hermeling, S., W. Jiskoot, et al. (2005), Pharmaceutical Research 22(6): 847-851; Hermeling, S., H. Schellekens, et al. (2006), Journal Of Pharmaceutical Sciences 95(5): 1084-1096).

Despite the knowledge that aggregates can lead to immune response, it is not trivial to remove aggregates that are present in therapeutic proteins. The process itself may induce aggregation. A review of myriad potential aggregation pathways during the production of protein therapeutics is provided by Chi, E. Y., S. Krishnan, et al. (2003), Protein Science 12(5): 903-913. Many aggregates can be removed through the judicial use of processing steps, however it is difficult to have 100% purity. There also exists incidents where a protein is surface active and aggregation is induced as the protein transfers across the membrane (Maa, Y. F. and C. C. Hsu (1998), Journal Of Pharmaceutical Sciences 87(7): 808-812). Aggregates in the process can also hinder downstream processing steps and result in lower product purity (Sin, S. C., H. Baldascini, et al. (2006), Bioprocess And Biosystems Engineering 28(6): 405-414).

High pressure treatment provides an effective process for the removal of protein aggregates because it does not involve filtration or purification that can induce aggregation. However, conditions must be identified that do not induce aggregation of the monomer (in any form) while still dissociating aggregates. One skilled in the art would expect to refold a solution comprising more than 90% aggregate to high yield. Contrary thereto, that condition will not be able to provide a solution containing low levels of aggregate when monomeric material is present initially and conditions must be practical for downstream processing solutions.

The current invention is directed, in part, to use of high pressure techniques to alleviate the problem of soluble aggregates in recombinant protein preparations, especially in preparations of recombinant proteins that are relatively high in monomer content, and to preparations of recombinant proteins substantially free of soluble aggregates.

Protein therapeutics with reduced immunogenicity would address, at least, some of these issues. Furthermore, a method for removal of soluble aggregates from protein therapeutics that, in turn, reduces immunogenicity would contribute significantly to the safety, increased bioactivity and increased efficacy of therapeutic proteins. Given this, and the current technologies known to those in the art, the process of reducing immunogenicity presents a dilemma to the industry. The present invention addresses these problems and provides advances and improvements in the art of recombinant protein therapeutics.

### SUMMARY OF THE INVENTION

The invention provides particularly effective and efficient methods for the reduction of immunogenicity in protein therapeutics, more specifically recombinant protein therapeutics. The methods provide routes for overcoming protein therapeutic immunogenicity and related difficulties by employing the use of high pressure treatment. These methods allow for the production of high quality recombinant protein therapeutic while circumventing problems that would otherwise be associated with bioactivity, efficacy, immunogenicity, and the like. The methods advantageously provide processing benefits and therapeutic benefits associated with recombinant proteins.

High pressure refolding has been identified to occur at conditions within a “pressure-window” that generally favors the native protein conformation. However, identifying conditions that completely stabilize the monomer is difficult, because some conditions for refolding solutions comprising greater than 90% aggregates will induce aggregation in monomeric solutions. Since high pressure has been shown to induce aggregate for monomeric material in many protein classes, this feature of the present invention is significant and novel (Ferrao-Gonzales, A. D., S. O, Souto, et al. (2000), Proceedings of the National Academy of Sciences of the United States of America 97(12): 6445-6450; Kim, Y. S., T. W. Randolph, et al. (2002), Journal of Biological Chemistry 277(30): 27240-27246; Seefeldt, M. B., Y. S. Kim, et al. (2005), Protein Science 14(9): 2258-2266; Dzwolak, W. (2006), Biochimica Et Biophysica Acta-Proteins And Proteomics 1764(3): 470-480; Grudzielanek, S., V. Smirnovas, et al. (2006), Journal Of Molecular Biology 356(2): 497-509; Kim, Y. S., T. W. Randolph, et al. (2006), High-pressure studies on protein aggregates and amyloid fibrils. Methods in Enzymology: Amyloid, Prions, And Other Protein Aggregates, Pt C. 413: 237-253). This invention identifies conditions which dissociate aggregates without aggregating any of the monomer.

In particular, the invention embraces methods of reducing protein aggregates in therapeutic protein preparations, and protein preparations treated with such methods. In one embodiment, the invention comprises a method of treating a protein preparation suspected of containing aggregates, comprising subjecting the protein preparation to high hydrostatic pressure for a period of time, and reducing the pressure to atmospheric pressure, wherein the protein preparation has reduced immunogenicity compared to the protein preparation before high-pressure treatment. In another embodiment, the protein preparation is a therapeutic protein preparation.

In many preparations of therapeutic proteins with high monomer content (for example, about 80% monomer or greater than about 80% monomer; about 90% monomer or greater than about 90% monomer; about 95% monomer or greater than about 95% monomer; about 98% monomer or greater than about 98% monomer) or relatively low aggregate content (for example, about 20% aggregate content or less than about 20% aggregate content; about 10% aggregate content or less than about 10% aggregate content; about 5% aggregate content or less than about 5% aggregate content; about 2% aggregate content or less than about 2% aggregate content), conditions for reducing aggregates must be chosen carefully, as an injudicious choice of refolding conditions can actually increase aggregate content. Thus, in one embodiment, the invention embraces methods of reducing aggregate content or increasing monomer content in a preparation of protein with high monomer content or low aggregate content, comprising subjecting the preparation to high-pressure conditions that do not induce aggregation, where the conditions include magnitude of high pressure, duration of high-pressure treatment, protein concentration, temperature, pH, ionic strength, chaotrope concentration, surfactant concentration, buffer concentration, preferential excluding compounds concentration, or other solution parameters as described herein. In one embodiment, the methods of reducing aggregate content or increasing monomer content in a preparation of protein with high monomer content or low aggregate content are performed after purification of the protein is completed, that is, after the protein is at the desired purity level for use as a therapeutic (where purity refers to undesired components besides the protein of interest, not to aggregates of the protein of interest).

In one embodiment of the invention, soluble aggregates in therapeutic protein preparations are reduced by at least about 10%, at least about 20%, at least about 25%, at least about 30%, at least about 40%, at least about 50%, at least about 75%, at least about 90%, at least about 95%, or at least about 99% when treated with high-pressure methods, as compared to a preparation of the same protein which is not treated with high-pressure methods. In another embodiment of the invention, soluble aggregates in therapeutic protein preparations are reduced to an undetectable level when treated with high-pressure methods, as compared to a preparation of the same protein which is not treated with high-pressure methods.

In another embodiment of the invention, an initial therapeutic protein preparation having a monomer content of at least about 80% is treated with the high-pressure methods of the invention to reduce soluble aggregates in the therapeutic protein preparation by at least about 10%, at least about 20%, at least about 25%, at least about 30%, at least about 40%, at least about 50%, at least about 75%, at least about 90%, at least about 95%, or at least about 99% when treated with high-pressure methods, as compared to the initial preparation of the protein prior to treatment with high-pressure methods. In another embodiment of the invention, soluble aggregates in an initial therapeutic protein preparation having a monomer content of at least about 80% are reduced to an undetectable level when treated with high-pressure methods, as compared to the initial preparation of the protein prior to treatment with high-pressure methods.

In another embodiment, the invention embraces a therapeutic protein preparation treated by high pressure, and a method of making a therapeutic protein preparation treated by high pressure, where the therapeutic protein preparation causes a reduced or undetectable immune response to the protein after administration of the protein composition to an individual in need thereof, as compared to the immune response to a preparation of the same protein which is not treated by high pressure. In one embodiment of the invention, the invention encompasses a therapeutic protein preparation treated by high pressure, and a method of making a therapeutic protein preparation treated by high pressure, where the immune response to the therapeutic protein preparation treated by high pressure is reduced by at least about 50% as compared to the immune response to a preparation of the same protein which is not treated by high pressure. In one embodiment, the only difference between the therapeutic protein preparation treated by high pressure and the preparation of the same protein which is not treated by high pressure is the pressure treatment itself, where the high-pressure treatment is conducted under conditions that reduce aggregate in a highly monomeric solution of protein (the highly monomeric solution comprising greater than or equal to about 90% monomer). In another embodiment of the invention, the immune response to the therapeutic protein preparation treated by high pressure is reduced by at least about 75% as compared to the immune response to a preparation of the same protein which is not treated by high pressure. In another embodiment of the invention, the immune response to the therapeutic protein preparation treated by high pressure is reduced by at least about 90% as compared to the immune response to a preparation of the same protein which is not treated by high pressure. In another embodiment of the invention, the immune response to the therapeutic protein preparation treated by high pressure is reduced by at least about 95% as compared to the immune response to a preparation of the same protein which is not treated by high pressure. In another embodiment of the invention, the immune response to the therapeutic protein preparation treated by high pressure is reduced by at least about 99% as compared to the immune response to a preparation of the same protein which is not treated by high pressure. In another embodiment of the invention, the immune response to a therapeutic protein preparation treated by high pressure is substantially undetectable compared to the immune response to a preparation of the same protein which is not treated by high pressure.

In another embodiment, the invention embraces a method of administering a therapeutic protein preparation of reduced immunogenicity, comprising subjecting a therapeutic protein preparation to high pressure for a period of time; releasing the pressure; and administering the therapeutic protein preparation to an individual. The high pressure can be between about 500 bar and about 10,000 bar, between about 500 bar and about 5000 bar, between about 1000 bar and about 3500 bar, between about 1000 bar and about 3000 bar, or at about 2000 bar. The pressure can be released at a controlled depressurization rate, such as between 10 bar/minute and 100 bar/minute. The therapeutic protein preparation is administered to the individual within about 24 hours, about 12 hours, about 4 hours, about 1 hour, or about 15 minutes of releasing the pressure. In some embodiments, the protein is endogenous to the species to which the individual belongs; in other embodiments, the protein is not endogenous to the species to which the individual belongs

In one embodiment, the invention embraces a protein composition, comprising a protein and a pharmaceutically acceptable carrier, wherein the protein composition is administered to an individual, wherein protein-specific antibody levels are substantially undetectable after administration. In another embodiment, the invention embraces a protein composition, comprising a protein and a pharmaceutically acceptable carrier, wherein the protein composition is administered to an individual, wherein protein-specific antibody levels are substantially the same as protein-specific antibody levels prior to protein administration. In another embodiment, the invention embraces a protein composition, comprising a protein and a pharmaceutically acceptable carrier, wherein the protein composition is administered to an individual, wherein protein-specific antibody levels are less than protein-specific antibody levels produced by administration of an aggregated protein composition. In some embodiments, the protein is endogenous to the species to which the individual belongs; in other embodiments, the protein is not endogenous to the species to which the individual belongs

In another embodiment, the invention embraces a protein composition, comprising a protein and a pharmaceutically acceptable carrier, wherein the protein composition contains less than about 20% of aggregated protein as a percentage of total protein, or wherein the protein composition contains less than about 10% of aggregated protein as a percentage of total protein, or wherein the protein composition contains less than about 5% of aggregated protein as a percentage of total protein, or wherein the protein composition contains less than about 1% of aggregated protein as a percentage of total protein, or wherein the protein composition contains no substantially detectable amount of aggregated protein as a percentage of total protein. The amount of aggregated protein in the protein composition is measured by any method including, but not limited to, analytical ultracentrifugation, size exclusion chromatography, field flow fractionation, light scattering, light obscuration, fluorescence spectroscopy, gel electrophoresis, GEMMA analysis, and nuclear magnetic resonance spectroscopy. The percentage can be based on any one method of analysis, to the exclusion of other methods of analysis. Alternatively, the amount of aggregated protein in the protein composition measured by at least one method, including, but not limited to, analytical ultracentrifugation, size exclusion chromatography, field flow fractionation, light scattering, light obscuration, fluorescence spectroscopy, gel electrophoresis, GEMMA analysis, and nuclear magnetic resonance spectroscopy. That is, the percentage can be based on any one method of analysis, without necessarily excluding other methods of analysis.

In another embodiment, the invention embraces a protein composition, comprising a protein and a pharmaceutically acceptable carrier, wherein the protein composition does not break immune tolerance of an individual to the protein.

In another embodiment, the invention embraces a protein and a pharmaceutically acceptable carrier, wherein the protein composition does not break the immune tolerance to the protein of a transgenic animal carrying a transgene encoding the protein.

In another embodiment, the invention embraces a protein and a pharmaceutically acceptable carrier, wherein the protein composition does not break the immune tolerance to the protein of an animal with induced tolerances to the protein.

The invention embraces a testing for reduced immunogenicity of a high-pressure treated protein to the same protein which has not been treated with high pressure, comprising a) subjecting a solution of the protein to high-pressure treatment; b) before or after step a, placing the high-pressure treated protein in a pharmaceutically acceptable carrier if it is not already in such a carrier; c) administering the high-pressure treated protein to a first individual; d) at any point in the method, placing the non-high-pressure treated protein in a pharmaceutically acceptable carrier if it is not already in such a carrier; e) at any point in the method after placing in a pharmaceutically acceptable carrier, administering the non-high-pressure treated protein to a second individual; and f) comparing the immune response of the first individual to the second individual; wherein a reduced immune response of the first individual as compared to the second individual indicates that the high-pressure treated protein has reduced immunogenicity. The immune response can be measured by antibody titers, relative or absolute amount of antibodies present, clinical immune reactions such as inflammation and reactions associated with anaphylaxis (weakness, itching, swelling, hives, cramps, diarrhea, vomiting, difficulty breathing, tightness in the chest, lowered blood pressure, loss of consciousness, and shock), amount of time required for a preparation to provoke detectable antibodies, amount of time required for a preparation to provoke a specified antibody titer, and amount of time required for a preparation to provoke a certain concentration level of antibody. The immune response can be measured by a Biacore assay. The first and second individuals can be transgenic animals, where the transgene expresses the protein used in the method, or the first and second individuals can be tolerized to the protein used in the method.

In any of the methods described above, the immune response can be measured by any suitable assay known to those of skill in the art, including antibody titers, relative or absolute amount of antibodies present, clinical immune reactions such as inflammation and reactions associated with anaphylaxis (weakness, itching, swelling, hives, cramps, diarrhea, vomiting, difficulty breathing, tightness in the chest, lowered blood pressure, loss of consciousness, and shock), amount of time required for a preparation to provoke detectable antibodies, amount of time required for a preparation to provoke a specified antibody titer, and amount of time required for a preparation to provoke a certain concentration level of antibody.

The methods and compositions of the invention allow for protein therapeutics that have reduced immunogenicity. In some modes of practice the methods are advantageously employed to provide improved methods for the production of recombinant protein therapeutics. The methods can provide such improvements as reduced immunogenicity in concert with increased bioactivity and/or increased efficacy, as well as improvement in the protein yield and/or quality.

### BRIEF DESCRIPTION OF THE FIGURES

FIG. 1 depicts the effect of high pressure and ionic strength on the refolding of >90% aggregated CTLA-4-Ig.

FIG. 2 depicts the effect of pressure and ionic strength on the stability of monomeric CTLA-4-Ig fusion proteins.

FIG. 3 depicts the effect of ionic strength and pressure on the refolding of solutions comprising moderate aggregate levels.

FIG. 4 depicts the antibody response to Nordiflex rhGH dosing in naïve mice (4th bleed) as a function of treatment level.

FIG. 5 depicts dissociation of IFN-beta aggregates through the use of high pressure. Aggregates were formed as a result of a modified version of the process taught by Shaked et al (Shaked, Stewart et al. 1993) (see Methods).

FIG. 6 depicts the ELISA response of naïve mice dosed with monomer, aggregated, and high pressure treated aggregates of rmIFN-beta. Dosing was conducted at either 0.5 ug/dose or 2.3 ug/dose for fifteen days.

### DETAILED DESCRIPTION OF THE INVENTION

All publications and patents mentioned herein are hereby incorporated by reference in their respective entireties. The publications and patents disclosed herein are provided solely for their disclosure. Nothing herein is to be construed as an admission that the inventors are not entitled to antedate any publication and/or patent, including any publication and/or patent cited herein.

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

The method of the present invention can be used to make recombinant proteins having reduced immunogenicity, and are especially useful for the removal of soluble aggregates therefrom.

More specifically, the methods described herein include steps for treating a protein under high pressure to reduce immunogenicity of the protein preparation, comprising the steps of subjecting a solution of the protein to high pressure, then reducing the pressure to ambient pressure. The conditions, which include the high pressure level chosen, temperature, pH, and other conditions as described herein, are chosen so as to dissociate soluble aggregates while not inducing further aggregation of the protein. This minimizes or eliminates the soluble aggregates of the protein and therefore improves the quality of the protein therapeutic.

By “high pressure” is meant a pressure of at least about 250 bar. The pressure at which the methods of the invention are used can be at least about 250 bar of pressure, at least about 400 bar of pressure, at least about 500 bar of pressure, at least about 1 kbar of pressure, at least about 2 kbar of pressure, at least about 3 kbar of pressure, at least about 5 kbar of pressure, or at least about 10 kbar of pressure.

As used herein, a “protein aggregate” is defined as being composed of a multiplicity of protein molecules wherein non-native noncovalent interactions and/or non-native covalent bonds (such as non-native intermolecular disulfide bonds) hold the protein molecules together. Typically, but not always, an aggregate contains sufficient molecules so that it is insoluble; such aggregates are insoluble aggregates. There are also proteins which form non-native aggregates that remain in solution; such aggregates are soluble aggregates. In addition, there is typically (but not always) a display of at least one epitope or region on the aggregate surface which is not displayed on the surface of native, non-aggregated protein. “Inclusion bodies” are a type of aggregate of particular interest to which the present invention is applicable. Other protein aggregates include, but are not limited to, soluble and insoluble precipitates, soluble non-native oligomers, gels, fibrils, films, filaments, protofibrils, amyloid deposits, plaques, and dispersed non-native intracellular oligomers.

“Atmospheric,” “ambient,” or “standard” pressure is defined as approximately 15 pounds per square inch (psi) or approximately 1 bar or approximately 100,000 Pascals.

“Biological activity” of a protein as used herein, means that the protein retains at least about 10% of maximal known specific activity as measured in an assay that is generally accepted in the art to be correlated with the known or intended utility of the protein. For proteins intended for therapeutic use, the assay of choice is one accepted by a regulatory agency to which data on safety and efficacy of the protein must be submitted. A protein having at least about 10% of maximal known specific activity is “biologically active” for the purposes of the invention.

“Denatured,” as applied to a protein in the present context, means that native secondary, tertiary, and/or quaternary structure is disrupted to an extent that the protein does not have biological activity.

In contrast to “denatured,” the “native conformation” of a protein refers to the secondary, tertiary and/or quaternary structures of a protein as it occurs in nature in its biologically active state.

“Tolerance” or “immune tolerant” as used herein, refers to the absence of an immune response to a specific antigen in the setting of an otherwise substantially normal immune system. Tolerance is distinct from generalized immunosuppression, in which all, or part of, immune responses are diminished.

“Transgenic animal” as used herein, refers to any non-human animal in which one or more of the cells of the animal contain nucleic acid received, directly or indirectly, by genetic manipulation such as microinjection or infection with a recombinant virus. The introduced nucleic acid may be integrated within a chromosome, or it may be extra-chromosomally replicating. The term “germ-line transgenic animal” refers to an animal in which the nucleic acid is introduced into a germ line cell, thereby conferring the ability to transfer the information to offspring. Such non-human animals include, but are not limited to, rodents, non-human primates, sheep, dogs, cows, goats, pigs and cats.

An “individual” means an animal with a functional immune system, such as a vertebrate, a bird, a mammal, or a human. The individual may be an experimental animal, such as an experimental mammal such as a rat, a mouse, or a rabbit. The individual may be a veterinary animal in need of therapy or treatment. The individual may be a human patient in need of therapy or treatment.

By “substantially the same” is meant that the difference in levels is less than about three, about two, or about one times the standard deviation in experimental measurement, preferably less than about one times the standard deviation. By “substantially undetectable” is meant that the difference between the zero or control measurement and the sample measurement is less than about three, about two, or about one times the standard deviation in experimental measurement, preferably less than about one times the standard deviation.

A “therapeutic protein preparation” is any composition comprising a protein, preferably a liquid composition comprising a protein, where the protein is intended to be used as a drug. A therapeutic protein preparation need not necessarily be in the final formulation for use as a drug; it can be in any formulation suitable for preparing or processing the protein, including, but not limited to, its final formulation for administration as a drug. The liquid in a liquid protein composition can be liquids including, but not limited to, water, a buffer, a pharmaceutically acceptable carrier, or a denaturant solution.

Considerations for Pressure Treatment to Remove Soluble Aggregates and other Immunogenic Species

Protein compositions which can be treated with the methods of the invention include, but are not limited to, laboratory samples, bulk pharmaceutical preparations, and individual dosages or individual dose units of the proteins. In one embodiment of the invention, a bulk pharmaceutical preparation of a protein is treated with high pressure prior to dividing the preparation into individual dosages, individual dose units or individual containers. This treatment can be performed at any time prior to use of the pharmaceutical, for example, at least about 3 years before the protein composition is intended to be administered to a individual, at least about 2 years before the protein composition is intended to be administered to a individual, at least about 1 year before the protein composition is intended to be administered to a individual, at least about 6 months before the protein composition is intended to be administered to a individual, at least about 3 months before the protein composition is intended to be administered to a individual, at least about 1 month before the protein composition is intended to be administered to a individual, at least about 2 weeks before the protein composition is intended to be administered to a individual, at least about 1 week before the protein composition is intended to be administered to a individual, at least about 3 days before the protein composition is intended to be administered to a individual, at least about 1 day before the protein composition is intended to be administered to a individual, at least about 12 hours before the protein composition is intended to be administered to a individual, at least about 4 hours before the protein composition is intended to be administered to a individual, at least about 1 hour before the protein composition is intended to be administered to a individual, or at least about 15 minutes before the protein composition is intended to be administered to a individual. Alternatively, the treatment can be performed at most about 3 years before the protein composition is intended to be administered to a individual, at most about 2 years before the protein composition is intended to be administered to a individual, at most about 1 year before the protein composition is intended to be administered to a individual, at most about 6 months before the protein composition is intended to be administered to a individual, at most about 3 months before the protein composition is intended to be administered to a individual, at most about 1 month before the protein composition is intended to be administered to a individual, at most about 2 weeks before the protein composition is intended to be administered to a individual, at most about 1 week before the protein composition is intended to be administered to a individual, at most about 3 days before the protein composition is intended to be administered to a individual, at most about 1 day before the protein composition is intended to be administered to a individual, at most about 12 hours before the protein composition is intended to be administered to a individual, at most about 4 hours before the protein composition is intended to be administered to a individual, at most about 1 hour before the protein composition is intended to be administered to a individual, or at most about 15 minutes before the protein composition is intended to be administered to a individual. One advantage of the pressure treatment is that the shelf life of a therapeutic protein preparation can often be extended, as removing aggregated and/or non-native species also removes nucleation sites for further aggregation and/or formation of non-native species, and thus slows the rate of such undesirable results. In another embodiment of the invention, the invention embraces a method of preparing a protein composition where the shelf life of the therapeutic protein preparation is increased by at least about 100% by high-pressure treatment of the therapeutic protein preparation, at least about 50% by high-pressure treatment of the therapeutic protein preparation, at least about 25% by high-pressure treatment of the therapeutic protein preparation, or at least about 10% by high-pressure treatment of the therapeutic protein preparation. In another embodiment of the invention, the invention embraces a pressure-treated protein composition with a shelf life which is increased by at least about 100%, at least about 50% by high-pressure treatment of the therapeutic protein preparation, at least about 25% by high-pressure treatment of the therapeutic protein preparation, or at least about 10% by high-pressure treatment of the therapeutic protein preparation

As noted above, a “therapeutic protein preparation” need not be in its final formulation for administration. In some instances, a commercial therapeutic protein preparation will be supplied in a formulation suitable for administration, but for purposes of removal of soluble aggregates and/or other non-native protein, the formulation can be changed to a formulation more suitable for removal of soluble aggregates and/or other non-native protein. Thus, for example, in order to treat a commercial therapeutic protein preparation (which is in a formulation suitable for administration) to remove soluble aggregates and/or other non-native protein, the formulation can be changed by altering the pH (for example, from a final formulation pH of 7 to a pH of 3), treating the protein preparation to remove soluble aggregates and/or other non-native protein, and then restoring the pH to a value suitable for administration. The therapeutic protein preparation can also be in a form recovered from “downstream processing,” that is, after various refolding and chromatographic or other purification steps which result in a highly-monomeric preparation of protein (for example, of greater than or equal to about 90% monomeric) which still contains substantial amounts of soluble aggregates. Other parameters, such as protein concentration, salt concentration, buffer concentration, temperature, and chaotrope concentration can be adjusted in such a manner.

Alternatively, a manufacturer may supply a therapeutic protein preparation in a formulation suitable for high-pressure treatment to remove soluble aggregates and/or other non-native proteins, and the therapeutic protein preparation can then be adjusted to comprise a formulation suitable for administration as a drug.

When performing comparative testing of the therapeutic protein preparation, the time that elapses after pressure treatment (i.e., after releasing the high pressure) and before administering the high-pressure treated protein preparation to a first individual can be any of the time periods as stated above before for high pressure treatment prior to use of a pharmaceutical.

### Proteins for Refolding

The invention embraces any protein where refolding is desired, such as recombinant proteins, proteins isolated from natural sources, or proteins produced by chemical synthesis. Specific proteins which can be treated with the methods of the invention include: interferon-alpha; interferon-alpha 2a (Roferon-A; Pegasys); interferon-beta 1b (Betaseron); interferon-beta 1a (Avonex); insulin (Humulin-R); DNAase (Pulmozyme); Neupogen; Epogen; Procrit (Epotein Alpha); Aranesp (2nd Generation Procrit); Intron A (interferon-alpha 2b); Rituxan (Rituximab anti-CD20); IL-2 (Proleukin); IL-1 ra (Kineret); BMP-7 (Osteogenin); TNF-alpha I a (Beromun); HUMIRA (anti-TNF-alpha MAB); tPA (Tenecteplase); PDGF (Regranex®); interferon-gamma 1b (Actimmune); uPA; GMCSF; Factor VIII; Remicade (infliximab); Enbrel (Etanercapt); Betaferon (interferon beta-1a); Saizen (somatotropin); Erbitux (cetuximab); Saizen (somatropin); Norditropin (somatropin); Nutropin (somatropin); Genotropin (somatropin); Humatrope (somatropin); Rebif (interferon beta 1a); Herceptin (trastuzumab); and Humira (adalimumab). Immunoglobulins (such as IgG) and other proteins can be treated with the methods of the invention as well.

### Protein Analysis

Several methods are available for analyzing and quantitating aggregated proteins. An excellent overview of several methods of analysis of macromolecules is found in Cantor, C. R. and P. R. Schimmel, Biophysical Chemistry Part II. Techniques for the Study of Biological Structure and Function, W.H. Freeman & Co., New York: 1980. Other general techniques are described in US Patent Application Publication No. 2003/0022243.

The use of analytical ultracentrifugation for characterization of aggregation of protein therapeutics is specifically discussed in Philo, J. S., American Biotechnology Laboratory, page 22, October 2003. Experiments that can be performed using analytical ultracentrifugation include sedimentation velocity and sedimentation equilibrium experiments, which can be performed to determine whether multiple solutes exist in a solution (e.g., monomer, dimer, trimer, etc.) and provide an estimate of molecular weights for the solutes.

Size-exclusion chromatography and gel permeation chromatography can be used to estimate molecular weights and aggregation numbers of proteins, as well as for separation of different aggregates. See references such as Wu, C.-S. (editor), Handbook of Size Exclusion Chromatography and Related Techniques, Second Edition (Chromatographic Science), Marcel Dekker: New York, 2004 (particularly chapter 15 at pages 439-462 by Baker et al., “Size Exclusion Chromatography of Proteins”) and Wu, C.-S. (editor), Column Handbook for Size Exclusion Chromatography, San Diego: Academic Press, 1999 (particularly Chapters 2 and 18).

Field flow fractionation, which relies on a field perpendicular to a liquid stream of molecules, can also be used to analyze and separate aggregated proteins such as protein monomers, dimers, trimers, etc. See Zhu et al., Anal. Chem. 77:4581 (2005); Litzen et al., Anal. Biochem. 212:469 (1993); and Reschiglian et al., Trends Biotechnol. 23:475 (2005).

Light scattering methods, such as methods using laser light scattering (often in conjunction with size-exclusion chromatography or other methods) can also be used to estimate the molecular weight of proteins, including protein aggregates; see, for example, Mogridge, J., Methods Mol. Biol. 261:113 (2004) and Ye, H., Analytical Biochem. 356:76 (2006). Dynamic light scattering techniques are discussed in Pecora, R., ed., Dynamic Light Scattering: Applications of Photon Correlation Spectroscopy, New York: Springer Verlag, 2003 and Berne, B. J. and Pecora, R., Dynamic Light Scattering: With Applications to Chemistry, Biology, and Physics, Mineola, N.Y.: Dover Publications, 2000. Laser light scattering is discussed in Johnson, C. S. and Gabriel, D. A., Laser Light Scattering, Mineola, N.Y.: Dover Publications, 1995, and other light scattering techniques which can be applied to determine protein aggregation are discussed in Kratochvil, P., Classical Light Scattering from Polymer Solutions, Amsterdam: Elsevier, 1987.

Light obscuration can also be used to measure protein aggregation; see Seefeldt et al., Protein Sci. 14:2258 (2005); Kim et al., J. Biol. Chem. 276: 1626 (2001); and Kim et al., J. Biol. Chem. 277: 27240 (2002).

Fluorescence spectroscopy, such as fluorescence anisotropy spectroscopy, can be used to determine the presence of protein aggregates. Fluorescence probes (dyes) can be covalently or non-covalently bound to the aggregate to aid in analysis of aggregates (see, e.g., Lindgren et al., Biophys. J. 88: 4200 (2005)), US Patent Application Publication 2003/0203403), or Royer, C. A., Methods Mol. Biol. 40:65 (1995). Internal tryptophan residues can also be used to detect protein aggregation; see, e.g., Dusa et al., Biochemistry 45:2752 (2006).

Many methods of gel electrophoresis can be employed to analyze proteins and protein aggregation. One of the most common methods of gel electrophoresis is polyacrylamide gel electrophoresis (PAGE). If an aggregate is covalently linked, denaturing PAGE (using, e.g., sodium dodecyl sulfate) can be employed. Native PAGE (non-denaturing PAGE) can be used to study non-covalently linked aggregates. See, e.g., Hermeling et al. J. Phar. Sci. 95:1084-1096 (2006); Kilic et al., Protein Sci. 12:1663 (2003); Westermeier, R., Electrophoresis in Practice: A Guide to Methods and Applications of DNA and Protein Separations 4^{th} edition, New York: John Wiley & Sons, 2005; and Hames, B. D. (Ed.), Gel Electrophoresis of proteins: A Practical Approach, 3^{rd} edition, New York: Oxford University Press, USA, 1998.

Gas-phase electrophoretic mobility molecular analysis (GEMMA) (see Bacher et al., J. Mass Spectrom. 36:1038 (2001), Kaufman et al., Anal. Chem. 68:1895 (1996) and Kaufman et al., Anal. Biochem. 259:195 (1998)), a combination of electrophoresis in the gas phase and mass spectrometry, provides another method of analyzing protein complexes and aggregates.

Nuclear magnetic resonance spectroscopic techniques can be used to estimate hydrodynamic parameters related to protein aggregation. See, for example, James, T. L. (ed.), Nuclear Magnetic Resonance of Biological Macromolecules, Part C, Volume 394: Methods in Enzymology, San Diego: Academic Press, 2005; James, T. L., Dotsch, V. and Schmitz, U. (eds.), Nuclear Magnetic Resonance of Biological Macromolecules, Part A (Methods in Enzymology, Volume 338) and Nuclear Magnetic Resonance of Biological Macromolecules, Part B (Methods in Enzymology, Volume 339), San Diego: Academic Press, 2001, and Mansfield, S. L. et al., J. Phys. Chem. B, 103:2262 (1999). Linewidths, correlation times, and relaxation times are among the parameters that can be measured to estimate tumbling time in solution, which can then be correlated with the state of protein aggregation. Electron paramagnetic resonance (EPR or ESR) can also be used to determine aggregation states; see, e.g., Squier et al., J. Biol. Chem. 263:9162 (1988).

In one embodiment, the invention embraces a therapeutic protein preparation with a reduced level of protein aggregates. In one embodiment of the invention, soluble aggregates in therapeutic protein preparations are reduced by at least about 10% when treated with high-pressure methods, as compared to a preparation of the same protein which is not treated with high-pressure methods. In another embodiment of the invention, soluble aggregates in therapeutic protein preparations are reduced by at least about 20% when treated with high-pressure methods, as compared to a preparation of the same protein which is not treated with high-pressure methods. In another embodiment of the invention, soluble aggregates in therapeutic protein preparations are reduced by at least about 25% when treated with high-pressure methods, as compared to a preparation of the same protein which is not treated with high-pressure methods. In another embodiment of the invention, soluble aggregates in therapeutic protein preparations are reduced by at least about 30% when treated with high-pressure methods, as compared to a preparation of the same protein which is not treated with high-pressure methods. In another embodiment of the invention, soluble aggregates in therapeutic protein preparations are reduced by at least about 40% when treated with high-pressure methods, as compared to a preparation of the same protein which is not treated with high-pressure methods. In another embodiment of the invention, soluble aggregates in therapeutic protein preparations are reduced by at least about 50% when treated with high-pressure methods, as compared to a preparation of the same protein which is not treated with high-pressure methods. In another embodiment of the invention, soluble aggregates in therapeutic protein preparations are reduced by at least about 75% when treated with high-pressure methods, as compared to a preparation of the same protein which is not treated with high-pressure methods. In another embodiment of the invention, soluble aggregates in therapeutic protein preparations are reduced by at least about 90% when treated with high-pressure methods, as compared to a preparation of the same protein which is not treated with high-pressure methods. In another embodiment of the invention, soluble aggregates in therapeutic protein preparations are reduced by at least about 95% when treated with high-pressure methods, as compared to a preparation of the same protein which is not treated with high-pressure methods. In another embodiment of the invention, soluble aggregates in therapeutic protein preparations are reduced by at least about 99% when treated with high-pressure methods, as compared to a preparation of the same protein which is not treated with high-pressure methods. In another embodiment of the invention, soluble aggregates in therapeutic protein preparations are reduced to a substantially undetectable level when treated with high-pressure methods, as compared to a preparation of the same protein which is not treated with high-pressure methods.

In one embodiment of the invention, analytical ultracentrifugation is used for the comparison of aggregates in pressure-treated and untreated samples. In another embodiment of the invention, size exclusion chromatography is used for the comparison of aggregates in pressure-treated and untreated samples. In another embodiment of the invention, field flow fractionation is used for the comparison of aggregates in pressure-treated and untreated samples. In another embodiment of the invention, light scattering analysis is used for the comparison of aggregates in pressure-treated and untreated samples. In another embodiment of the invention, light obscuration analysis is used for the comparison of aggregates in pressure-treated and untreated samples. In another embodiment of the invention, fluorescence spectroscopy is used for the comparison of aggregates in pressure-treated and untreated samples. In another embodiment of the invention, gel electrophoresis is used for the comparison of aggregates in pressure-treated and untreated samples. In another embodiment of the invention, GEMMA is used for the comparison of aggregates in pressure-treated and untreated samples. In another embodiment of the invention, nuclear magnetic resonance spectroscopy is used for the comparison of aggregates in pressure-treated and untreated samples. In another embodiment of the invention, electron paramagnetic resonance spectroscopy is used for the comparison of aggregates in pressure-treated and untreated samples.

### Methods for Determining Immunogenicity

Immunogenicity of recombinant proteins may be evaluated in animal models. These models include naïve animals that do not express the recombinant protein of interest, but have been shown to have a stronger immune response to aggregate relative to monomer (Braun et al.) These models also include animals that have been induced to become tolerant to a specific antigen or transgenic animals that have been produced to carry a specific transgene and which are immune tolerant to the specific protein that is encoded by the transgene.

Induction of tolerance: Numerous strategies have been developed to induce antigen specific tolerance in animal models, for example with respect to autoimmune disorders, such as multiple sclerosis (or experimental allergic encephalitis, EAE) or diabetes, as well as to prevent rejection of allogeneic tissue transplants. The major methods developed in mouse and rat models involve administration of high doses of soluble antigen, oral ingestion of antigens or intrathymic injection. The efficacy of these methods depends to varying degrees on clonal deletion, clonal anergy, active suppression by antigen-specific T cells and immune deviation from cellular to humoral immune responses. See, for example, Friedman et al. PNAS 91:6688-6692 (1994); Higgins et al. J. Immunol. 140:440 (1988); Meyer et al. J. Immunol. 157:4230 (1996).

Tolerance can be developed in animals, for example, in mice or rats, by exposing the immature immune system to an antigen. Exposure of neonatal rodents to antigens to induce tolerance is well-known in the art. See also Burtles, S. S, and Hooper, D. C., Immunology 75:311 (1992); Yamaguchi et al., Journal of Immunological Methods 181:115 (1995); Forsthuber et al., Science 271:1728 (1996); Maverakis et al., J. Exp. Med. 191:695 (2000); Kramar et al., Journal of Autoimmunity 8:177 (1995); Kruisbeek et al., Journal of Experimental Medicine, 161:1029 (1985); and Cobbold, S. P., Phil. Trans. R. Soc. B 360, 1695 (2005).

Oral tolerance in animals, for example, mice or rats, may be induced by administrations of a protein either by a single feeding at a high dose or by a number of intermittent feedings of a small dose given on alternate days for a selected period of time. Animals are then tested for tolerance using standard methods known to those skilled in the art.

Antigen specific immune tolerance can also be induced in an animal by administration of an antigen in combination with a regimen of immunosuppression for a period of time sufficient to render the host tolerant to the antigen. Immunosuppression is accomplished by administration of an immunosuppressive agent. After a schedule of antigen administration and immunosuppression, the animal is capable of maintaining a specific immune tolerance to the antigen, even when the immunosuppressive agent is withdrawn. See, for example, U.S. Patent Application Publication No. 2004/0009906, and Cobbold, S. P., Phil. Trans. R. Soc. B 360, 1695 (2005).

Immune Tolerant Transgenic Animals: Transgenic animals may also be used to study immune tolerance to heterologous proteins. The transgenic animal carries a nucleic acid or “transgene” encoding a specific heterologous protein which makes the animal immunologically tolerant to the protein. Transgenic animals, usually transgenic mice, are available through commercial suppliers or other channels or may be produced as needed. See, for example, U.S. Pat. No. 5,470,560; Hermeling et al. J. Phar. Sci. 95:1084-1096 (2006); Hermeling et al. Pharm. Res. 22:847-851 (2005); Whiteley et al. J. Clin. Invest. 84:1550-1554 (1989).

A transgene may be foreign to the animal species, foreign only to the particular individual recipient or animal strain, or may be a variant of nucleic acid material or gene already possessed by the recipient. A transgene may be obtained by any method known by those skilled in the art, for example, by isolation from genomic sources, by preparation of cDNA from isolated mRNA templates, by directed synthesis, or by combinations thereof. A transgene should be operatively linked to a promoter in a functional manner for expression. Promoters and other regulatory elements may be used to increase, decrease, regulate or restrict to a specific tissue expression of the transgene. A promoter need not be the natural promoter associated with the transgene, and often is a promoter isolated from the recipient animal.

Transgenic animals may be produced by introducing a transgene into a germline cell of the recipient animal. The methods for introduction of genetic material into cells are generally available and well-known to those skilled in the art. Several methods that are commonly used include microinjection, retroviral infection, retroviral transduction and DNA transfection. See, for example, Gordon et al. PNAS 77:7380-7384 (1980); Hammer et al. J. Animal Sci. 63:269-278 (1986); Nagy et al. PNAS 90:8424-8428 (1993).

Transgenic animals carrying genetic material that expresses a heterologous protein should be immunologically tolerant to the protein, as the animal's immune system should recognize the heterologous protein as “self”. Thus transgenic animals can serve as models for studying immune tolerance and the immunogenicity of specific proteins, particularly proteins in aggregated and disaggregated states/formulations.

After production of a transgenic line carrying a specific transgene, the animals are screened for the presence of the heterologous polypeptide in serum or other body fluid. The polypeptide need not be produced in elevated levels or even at the levels of any endogenous homolog; the animal need only have produced sufficient polypeptide during maturation of the immune system so that the animal is tolerant to the polypeptide. Most commonly, tolerance is demonstrated by the observation that the animal is incapable of producing antibodies to the polypeptide when the polypeptide is administered to the animal.

The immune tolerant transgenic animal may be used to assess immunogenicity of a protein in different formulations or aggregated/disaggregated states. As a control, non-transgenic animals of the same genetic background as the transgenic animals should be included in the experiments. To test immunogenicity, non-transgenic or transgenic animals immune tolerant to the protein are injected with an heterologous protein. The animals may be injected by any route, including but not limited to, intraperitoneally (i.p.), intramuscularly (i.m.), subcutaneously (s.c.) or intravenously (i.v.). The animals may be injected according to a specific schedule, for example, days 1, 7, 14, 21 and 28, or days 1, 3, 7, 10, 14, 17 and 21 or days 1-4, 7-11 and 14-18. Serum samples are taken prior to any injections and at specific intervals thereafter, for example, weekly and 3 or 7 days after the last injection.

To demonstrate that a transgenic animal is immunologically responsive and tolerant only to the transgene encoded protein, an animal may be injected with an unrelated protein, such as human serum albumin or ovalbumin using the same injection schedule as used for the test protein. A reaction to such a foreign protein serves as a positive control indicating that the immune system of the transgenic animal is functioning normally.

Serum from the non-transgenic and transgenic animals may be evaluated for the presence of specific antibodies against the particular protein using conventional assays known to one skilled in the art. These assays include, but are not limited to, radioimmunoassays, enzyme-linked immunosorbent assays (ELISA) and surface plasmon resonance (SPR, e.g. BIACORE; BIACORE is a registered trademark of Biacore AB Corp., Uppsala, Sweden, for analyzers for measuring and investigating the interactions of biomolecules). A standard indirect ELISA technique is briefly described as an illustrative example. The test protein is diluted to a concentration of 2-10 μg/ml in a buffer such as PBS, TBS or carbonate-bicarbonate. 96-well plates are filled with 100 ul/well of the test protein and incubated overnight at 4° C. Plates are washed several times with a wash buffer (e.g., 0.05-0.1% Tween-20 in PBS). Unoccupied sites in wells are blocked by adding 200-300 ul/well of a blocking solution (e.g., 1-5% bovine serum albumin (BSA) in PBS) for 1 hour at room temperature. The plates are washed with wash buffer and serum samples from a mouse injected with the test protein are added to the wells in triplicate (50-100 ul/well). Plates are incubated for 1 hour at room temperature and subsequently washed three times. 100 μl enzyme-labeled anti-mouse IgG conjugate is added to each well and the plates are incubated for 1 hour at room temperature. Plates are washed and 100 μl of buffer containing an appropriate substrate is added to each well. After an incubation time for color development, absorbance is read in a microplate reader at a wavelength appropriate for the substrate used.

Systems based on surface plasmon resonance (SPR) offer detection and characterization of an immune response in serum samples. SPR can provide information on antibody isotype, specificity, kinetic profiles and affinity. Further, SPR has been shown to reliably detect low affinity antibodies which can be missed by other immunoassays. General information about BIACORE is provided in Nagata, K. and Handa, H. (eds.), Real-Time Analysis of Biomolecular Interactions Applications of Biacore, Tokyo: Springer-Verlag, 2000. Specific examples of the use of surface plasmon resonance (BIACORE) to detect antibodies are found in Kure et al., Intern. Med. 44: 100 (2005) (antibodies to insulin) and Mason et al., Curr. Med. Res. Opin. 19:651 (2003) (antibodies to erythropoietic molecules).

In one embodiment of the invention, the invention encompasses a therapeutic protein preparation treated by high pressure, and a method of making a therapeutic protein preparation treated by high pressure, where the immune response to the therapeutic protein preparation treated by high pressure is reduced by at least about 50% as compared to the immune response to a preparation of the same protein which is not treated by high pressure. In a preferred embodiment, the only difference between the therapeutic protein preparation treated by high pressure and the preparation of the same protein which is not treated by high pressure is the pressure treatment itself. In another embodiment of the invention, the immune response to the therapeutic protein preparation treated by high pressure is reduced by at least about 75% as compared to the immune response to a preparation of the same protein which is not treated by high pressure. In another embodiment of the invention, the immune response to the therapeutic protein preparation treated by high pressure is reduced by at least about 90% as compared to the immune response to a preparation of the same protein which is not treated by high pressure. In another embodiment of the invention, the immune response to the therapeutic protein preparation treated by high pressure is reduced by at least about 95% as compared to the immune response to a preparation of the same protein which is not treated by high pressure. In another embodiment of the invention, the immune response to the therapeutic protein preparation treated by high pressure is reduced by at least about 99% as compared to the immune response to a preparation of the same protein which is not treated by high pressure. In another embodiment of the invention, the immune response to a therapeutic protein preparation treated by high pressure is substantially undetectable compared to the immune response to a preparation of the same protein which is not treated by high pressure.

The immune response can be measured by any method known to those of skill in the art, including, but not limited to, antibody titers, relative or absolute amount of antibodies present, clinical immune reactions such as inflammation and reactions associated with anaphylaxis (weakness, itching, swelling, hives, cramps, diarrhea, vomiting, difficulty breathing, tightness in the chest, lowered blood pressure, loss of consciousness, and shock), amount of time required for a preparation to provoke detectable antibodies, amount of time required for a preparation to provoke a specified antibody titer, and amount of time required for a preparation to provoke a certain concentration level of antibody. As one of skill in the art will recognize, an improvement in an immune response where undesirable antibodies are generated will be a decreased amount of antibodies, or an increase in the amount of time required to generate undesirable antibodies.

The following examples are provided as exemplary calculations for calculating percentage reduction in immune response. For the immune response to a therapeutic protein preparation treated by high pressure to be reduced by, for example, at least about 75% as compared to the immune response to a preparation of the same protein which is not treated by high pressure, when using antibody levels as a comparison, the level of antibodies generated in response to the therapeutic protein preparation treated by high pressure would be only at most about 25% as compared to level of antibodies generated in response to a preparation of the same protein which is not treated by high pressure. When using time to provoke a given level of antibody production as a measurement of the immune response, if a given level is provoked in, for example, about 3 months by the preparation of the protein which is not treated by high pressure, then a reduction of at least about 75% reduction in the immune response can mean either 1) at 3 months, the level of antibodies provoked by the therapeutic protein preparation treated by high pressure is only at most about 25% of the level provoked by the preparation of the protein which is not treated by high pressure; or 2) the same level of antibody provoked in about 3 months by the preparation of the protein which is not treated by high pressure is provoked by the therapeutic protein preparation treated by high pressure in at least about 12 months (that is, a reduction in the immune response results in a lengthening of the time to provoke the same level of antibodies) (for time measurements, reducing the response by at least about (X) % is equivalent to lengthening the time by a factor of at least about (100 divided by (100−X)), so reducing the time response by 75% is equivalent to lengthening the time by a factor of (100/(100−75)=100/25, or a factor of 4); or both 1) and 2).

The immune response of an individual may be measured after a single administration of the therapeutic protein preparation. The immune response of an individual may also be measured after multiple administrations of the therapeutic protein preparation, such as after two administrations, after three administrations, after about 5 or more than about 5 administrations, after about 10 or more than about 10 administrations, after about 20 or more than about 20 administrations, after about 30 or more than about 30 administrations, after about 50 or more than about 50 administrations, after about 75 or more than about 75 administrations, or after about 100 or more than 100 administrations. Alternatively, the immune response of an individual may be measured after any duration of time, such as about after a week or more after, two weeks or more after, three weeks or more after, one month or more after, two months or more after, three months or more after, four months or more after, six months or more after, nine months or more after, twelve months or more after, eighteen months or more after, or twenty-four months or more after, one or multiple administrations of the therapeutic protein preparation.

### Other Considerations

Several conditions can be adjusted for optimal treatment of the protein preparation to reduce immunogenicity. Proteins can be treated by high pressure by placing them in a vessel (which can be a high-pressure variable-volume loading device) and then placing the vessel in a high-pressure generator, such as those available from High Pressure Equipment Co., Erie, Pa. High-pressure techniques are described in U.S. Pat. Nos. 6,489,450 and 7,064,192, U.S. Patent Application Publication No. 2004/0038333, and International Patent Application WO 02/062827; the methods for generating high pressure described therein are hereby incorporated by reference herein in their entirety. Certain devices have also been developed which are particularly suitable for refolding of proteins under high pressure; see International Patent Application Publication No. WO 2007/062174, which is incorporated by reference herein in its entirety. Some of the conditions which can be adjusted are described below.

Protein Concentration: the concentration of protein can be adjusted for optimal reduction in immunogenicity. Protein concentrations of at least about 0.1 mg/ml, at least about 1.0 mg/ml, at least about 5.0 mg/ml, at least about 10 mg/ml, or at least about 20 mg/ml can be used. Protein in the mixture may be present in a concentration of from about 0.001 mg/ml to about 300 mg/ml. Thus, in some embodiments the protein is present in a concentration of from about 0.001 mg/ml to about 250 mg/ml, from about 0.001 mg/ml to about 200 mg/ml, from about 0.001 mg/ml to about 150 mg/ml, from about 0.001 mg/ml to about 100 mg/ml, from about 0.001 mg/ml to about 50 mg/ml, from about 0.001 mg/ml to about 30 mg/ml, from about 0.05 mg/ml to about 300 mg/ml, from about 0.05 mg/ml to about 250 mg/ml, from about 0.05 mg/ml to about 200 mg/ml, from about 0.05 mg/ml to about 150 mg/ml, from about 0.05 mg/ml to about 100 mg/ml, from about 0.05 mg/ml to about 50 mg/ml, from about 0.05 mg/ml to about 30 mg/ml, from about 10 mg/ml to about 300 mg/ml, from about 10 mg/ml to about 250 mg/ml, from about 10 mg/ml to about 200 mg/ml, from about 10 mg/ml to about 150 mg/ml, from about 10 mg/ml to about 100 mg/ml, from about 10 mg/ml to about 50 mg/ml, from about 10 mg/ml to about 30 mg/ml, from about 0.1 mg/ml to about 100 mg/ml, from about 0.1 mg/ml to about 10 mg/ml, from about 1 mg/ml to about 100 mg/ml, from about 1 mg/ml to about 10 mg/ml, from about 10 mg/ml to about 100 mg/ml, or from about 50 mg/ml to about 100 mg/ml can be used.

As used in the present context the phrase “a period of time” and cognates thereof refer to the time needed to treat the protein preparation under high pressure to reduce immunogenicity. Typically, the times are about 15 minutes to about 50 hours, or possibly longer depending on the particular protein, (e.g., as long as necessary for the protein; for example, up to about 1 week, about 5 days, about 4 days, about 3 days, etc.). Thus, in some embodiments of the methods, the time sufficient for treatment of the protein preparation may be from about 2 to about 30 hours, from about 2 to about 24 hours, from about 2 to about 18 hours, from about 1 to about 10 hours, from about 1 to about 8 hours, from about 1 to about 6 hours, from about 2 to about 10 hours, from about 2 to about 8 hours, from about 2 to about 6 hours, or about 2 hours, about 6 hours, about 10 hours, about 16 hours, about 20 hours, or about 30 hours, from about 2 to about 10 hours, from about 2 to about 8 hours, from about 2 to about 6 hours, from about 12 to about 18 hours, or from about 10 to about 20 hours.

The protein preparation is typically in an aqueous solution. The protein preparation may also include other components, which may be present in the protein preparation, or which may be added to the protein preparation. These additional components may be one or more additional agents including: one or more stabilizing agents, one or more buffering agents, one or more surfactants, one or more disulfide shuffling agent pairs, one or more salts, one or more chaotropes, or combinations of two or more of the foregoing. When the protein preparation is to be used as a pharmaceutical and an additional component is added to the preparation, the component should either be pharmaceutically acceptable, or if not pharmaceutically acceptable, the added component should be removable from the protein preparation prior to administration as a pharmaceutical. For example, chaotropes such as urea can be removed by dialysis.

The amounts of the additional agents will vary depending on the selection of the protein, however, the effect of the presence (and amount) or absence of each additional agent or combinations of agents can be determined and optimized using the teachings provided herein.

Exemplary additional agents include, but are not limited to, buffers (examples include, but are not limited to, phosphate buffer, borate buffer, carbonate buffer, citrate buffer, HEPES, MEPS), salts (examples include, but are not limited to, the chloride, sulfate, and carbonate salts of sodium, zinc, calcium, ammonium and potassium), chaotropes (examples include, but are not limited to, urea, guanidine hydrochloride, guanidine sulfate and sarcosine), and stabilizing agents (e.g., preferential excluding compounds, etc.).

Non-specific protein stabilizing agents act to favor the most compact conformation of a protein. Such agents include, but are not limited to, one or more free amino acids, one or more preferentially excluding compounds, trimethylamine oxide, cyclodextrans, molecular chaperones, and combinations of two or more of the foregoing.

Amino acids can be used to prevent reaggregation and facilitate the dissociation of hydrogen bonds. Typical amino acids that can be used, but not limited to, are arginine, lysine, proline, glycine, histidine, and glutamine or combinations of two or more of the foregoing. In some embodiments, the free amino acid(s) is present in a concentration of about 0.1 mM to about the solubility limited of the amino acid, and in some variations from about 0.1 mM to about 2 M. The optimal concentration is a function of the desired protein and should favor the native conformation. Preferentially excluding compounds can be used to stabilize the native conformation of the protein of interest. Possible preferentially excluding compounds include, but are not limited to, sucrose, hexylene glycol, sugars (e.g., sucrose, trehalose, dextrose, mannose), and glycerol. The range of concentrations that can be use are from 0.1 mM to the maximum concentration at the solubility limit of the specific compound. The optimum preferential excluding concentration is a function of the protein of interest.

In particular embodiments, the preferentially excluding compound is one or more sugars (e.g., sucrose, trehalose, dextrose, mannose or combinations of two or more of the foregoing). In some embodiments, the sugar(s) is present in a concentration of about 0.1 mM to about the solubility limit of the particular compound. In some embodiments, the concentration is from about 0.1 mM to about 2M, from about 0.1 mM to about 1.5M, from about 0.1 mM to about 1M, from about 0.1 mM to about 0.5M, from about 0.1 mM to about 0.3M, from about 0.1 mM to about 0.2 M, from about 0.1 mM to about 0.1 mM, from about 0.1 mM to about 50 mM, from about 0.1 mM to about 25 mM, or from about 0.1 mM to about 10 mM.

In some embodiments, the stabilizing agent is one or more of sucrose, trehalose, glycerol, betaine, amino acid(s), or trimethylamine oxide.

In certain embodiments, the stabilizing agent is a cyclodextran. In some embodiments, the cyclodextran is present in a concentration of about 0.1 mM to about the solubility limit of the cyclodextran. In some variations from about 0.1 mM to about 2 M.

In certain embodiments, the stabilizing agent is a molecular chaperone. In some embodiments, the molecular chaperone is present in a concentration of about 0.01 mg/ml to 10 mg/ml.

A single stabilizing agent maybe be used or a combination of two or more stabilizing agents (e.g., at least two, at least three, or 2 or 3 or 4 stabilizing agents). Where more than one stabilizing agent is used, the stabilizing agents may be of different types, for example, at least one preferentially excluding compound and at least one free amino acid, at least one preferentially excluding compound and betaine, etc.

Buffering agents may be present to maintain a desired pH value or pH range. Numerous suitable buffering agents are known to the skilled artisan and should be selected based on the pH that favors (or at least does not disfavor) the native conformation of the protein of interest. Either inorganic or organic buffering agents may be used. Suitable concentrations are known to the skilled artisan and should be optimized for the methods as described herein according to the teaching provided based on the characteristics of the desired protein.

Thus, in some embodiments, at least one inorganic buffering agent is used (e.g., phosphate, carbonate, etc.). In certain embodiments, at least one organic buffering agent is used (e.g., citrate, acetate, Tris, MOPS, MES, HEPES, etc.) Additional organic and inorganic buffering agents are well known to the art.

In some embodiments, the one or more buffering agents is phosphate buffer, borate buffer, carbonate buffer, citrate buffer, HEPES, MEPS, MOPS, MES, or acetate buffer. In some embodiments, the one or more buffering agents is phosphate buffers, carbonate buffers, citrate, Tris, MOPS, MES, acetate or HEPES. A single buffering agent maybe be used or a combination of two or more buffering agents (e.g., at least two, at least 3, or 2 or 3 or 4 buffering agents).

A “surfactant” as used in the present context is a surface active compound which reduces the surface tension of water.

Surfactants are used to improve the solubility of certain proteins. Surfactants should generally be used at concentrations above or below their critical micelle concentration (CMC), for example, from about 5% to about 20% above or below the CMC. However, these values will vary dependent upon the surfactant chosen, for example, surfactants such as, beta-octylgluco-pyranoside may be effective at lower concentrations than, for example, surfactants such as TWEEN-20 (polysorbate 20). The optimal concentration is a function of each surfactant, which has its own CMC.

Useful surfactants include nonionic (including, but not limited to, t-octylphenoxypolyethoxy-ethanol and polyoxyethylene sorbitan), anionic (e.g., sodium dodecyl sulfate) and cationic (e.g., cetylpyridinium chloride) and amphoteric agents. Suitable surfactants include, but are not limited to deoxycholate, sodium octyl sulfate, sodium tetradecyl sulfate, polyoxyethylene ethers, sodium cholate, octylthioglucopyranoside, n-octylglucopyranoside, alkyltrimethylanmonium bromides, alkyltrimethyl ammonium chlorides, non-detergent sulfobetaines, and sodium bis(2 ethylhexyl) sulfosuccinate. In some embodiments the surfactant may be polysorbate 80, polysorbate 20, sarcosyl, Triton X-100, β-octyl-gluco-pyranoside, or Brij 35.

In some embodiments the one or more surfactant may be a polysorbate, polyoxyethylene ether, alkyltrimethylammonium bromide, pyranosides or combination of two or more of the foregoing. In certain embodiments, the one or more surfactant may be O-octyl-gluco-pyranoside, Brij 35, or a polysorbate.

In certain embodiments the one or more surfactant may be octyl phenol ethoxylate, β-octyl-gluco-pyranoside, polyoxyethyleneglycol dodecyl ether, sarcosyl, sodium dodecyl sulfate, polyethoxysorbitan, deoxycholate, sodium octyl sulfate, sodium tetradecyl sulfate, sodium cholate, octylthioglucopyranoside, n-octylglucopyranoside, sodium bis(2-ethylhexyl) sulfosuccinate or combinations of two or more of the foregoing. A single surfactant maybe be used or a combination of two or more surfactants (e.g., at least two, at least 3, or 2 or 3 or 4 surfactants).

Where the desired protein contains disulfide bonds in the native conformation it is generally advantageous to include at least one disulfide shuffling agent pair in the mixture. The disulfide shuffling agent pair facilitates the breakage of strained non-native disulfide bonds and the reformation of native-disulfide bonds. Disulfide shuffling agents can be removed by dialysis.

In general, the disulfide shuffling agent pair includes a reducing agent and an oxidizing agent. Exemplary oxidizing agents oxidized glutathione, cystine, cystamine, molecular oxygen, iodosobenzoic acid, sulfitolysis and peroxides. Exemplary reducing agents include glutathione, cysteine, cysteamine, diothiothreitol, dithioerythritol, tris(2-carboxyethyl)phosphine hydrochloride, or β-mercaptoethanol.

Exemplary disulfide shuffling agent pairs include oxidized/reduced glutathione, cystamine/cysteamine, and cysteine/cysteine.

Additional disulfide shuffling agent pairs are described by Gilbert H F. (1990). “Molecular and Cellular Aspects of Thiol Disulfide Exchange.” Advances in Enzymology and Related Areas of Molecular Biology 63:69-172, and Gilbert H F. (1995). “Thiol/Disulfide Exchange Equilibria and Disulfide Bond Stability.” Biothiols, Pt A. p 8-28, which are hereby incorporated by reference in their entirety.

The selection and concentration of the disulfide shuffling agent pair will depend upon the characteristics of the desired protein. Typically concentration of the disulfide shuffling agent pair taken together (including both oxidizing and reducing agent) is from about 0.1 mM to about 100 mM of the equivalent oxidized thiol, however, the concentration of the disulfide shuffling agent pair should be adjusted such that the presence of the pair is not the rate limiting step in disulfide bond rearrangement.

In some embodiments, the concentration will be about 1 mM, about 2 mM, about 3 mM about 5 mM, about 8 mM, about 9 mM, about 10 mM, about 20 mM, about 30 mM, about 40 mM, about 50 mM, about 60 mM, about 70 mM, about 80 mM, about 90 mM, about 100 mM, or from about 80 mM to about 100 mM, from about 0.1 mM to about 20 mM, from about 10 mM to about 50 mM, from about 1 mM to about 100 mM, from about 50 mM to about 100 mM, from about 20 mM to about 100 mM, from about 0.1 mM to about 10 mM, from about 0.1 mM to about 8 mM; from about 0.1 mM to about 6 mM, from about 0.1 mM to about 7 mM, from about 0.1 mM to about 5 mM, from about 0.1 mM to about 3 mM, from about 0.1 mM to about 1 mM.

A single disulfide shuffling agent pair maybe be used or a combination of two or more disulfide shuffling agent pairs (e.g., at least two, at least 3, or 2 or 3 or 4 disulfide shuffling agent pairs).

Chaotropic agents (also referred to as a “chaotrope”) are compounds, including, without limitation, guanidine, guanidine hydrochloride (guanidinium hydrochloride, GdmHCl), guanidine sulfate, urea, sodium thiocyanate, and/or other compounds which disrupt the noncovalent intermolecular bonding within the protein, permitting the polypeptide chain to assume a substantially random conformation

Chaotropic agents may be used in concentration of from about 10 mM to about 8 M. The optimal concentration of the chaotropic agent will depend on the desired protein as well as on the particular chaotropes selected. The choice of particular chaotropic agent and determination of optimal concentration can be optimized by the skilled artisan in view of the teachings provided herein. Chaotropes can be removed from protein preparations by, for example, dialysis before using the protein preparation as a pharmaceutical.

In some embodiments, the concentration of the chaotropic agent will be, for example, from about 10 mM to about 8 M, from about 10 mM to about 7 M, from about 10 mM to about 6 M, from about 0.1 M to about 8 M, from about 0.1 M to about 7 M, from about 0.1 M to about 6 M, from about 0.1 M to about 5 M, from about 0.1 M to about 4 M, from about 0.1 M to about 3 M, from about 0.1 M to about 2 M, from about 0.1 M to about 1 M, from about 10 mM to about 4 M, from about 10 mM to about 3 M, from about 10 mM to about 2 M, from about 10 mM to about 1 M, or about, 10 mM, about 50 mM, about 75 mM, about 0.1 M, about 0.5 M, about 0.8 M, about 1 M, about 2 M, about 3 M, about 4 M, about 5 M, about 6 M, about 7 M, about 8 M.

When used in the present methods, it is often advantageous to use chaotropic agents in non-denaturing concentrations to facilitate the dissociation of hydrogen bonds. While a non-denaturing concentration will vary depending on the desired protein, the range of non-denaturing concentrations is typically from about 0.1 to about 4 M. In some embodiments the concentration is from about 0.1 M to about 2 M.

In certain embodiments, guanidine hydrochloride or urea are the chaotropic agents. A single chaotropic agent maybe be used or a combination of two or more chaotropic agents (e.g., at least two, at least 3, or 2 or 3 or 4 chaotropic agents).

Agitation: Protein solutions can be agitated before and/or during refolding. Agitation can be performed by methods including, but not limited to, ultrasound energy (sonication), mechanical stirring, mechanical shaking, pumping through mixers, or via cascading solutions.

Temperature: The methods described herein can be performed at a range of temperature values, depending on the particular protein of interest. The optimal temperature, in concert with other factors, can be optimized as described herein. Proteins can be refolded at various temperatures, including at about room temperature, about 25° C., about 30° C., about 37° C., about 50° C., about 75° C., about 100° C., about 125° C., or ranges of from about 20 to about 125° C., about 25 to about 125° C., about 25 to about 100° C., about 25 to about 75° C., about 25 to about 50° C., about 50 to about 125° C., about 50 to about 100° C., about 50 to about 75° C., about 75 to about 125° C., about 5 to about 100° C., or about 100 to about 125° C.

In some embodiments of the methods, the temperature can range from about −20° C. to about 100° C. without adversely affecting the protein of interest, provided that prior to return to room temperature, the mixture is brought to a temperature at which it will not freeze. Thus in certain embodiments, the temperature may be from about 0° C. to about 75° C., from about 0° C. to about 55° C., from about 0° C. to about 35° C., from about 0° C. to about 25° C., from about 20° C. to about 75° C., from about 20° C. to about 65° C., from about 20° C. to about 35° C., from about 20° C. to about 25° C.

Although increased temperatures are often used to cause aggregation of proteins, when coupled with increased hydrostatic pressure it has been found that increased temperatures can enhance refolding recoveries effected by high pressure treatment, provided that the temperatures are not so high as to cause irreversible denaturation. Generally, the increased temperature for refolding should be about 20° C. lower than the temperatures at which irreversible loss of activity occurs. Relatively high temperatures (for example, about 60° C. to about 125° C., about 80° C. to about 110° C., including about 100° C., about 105° C., about 110° C., about 115° C., about 120° C. and about 125° C.) may be used while the solution is under pressure, as long as the temperature is reduced to a suitably low temperature before depressurizing. Such a suitably low temperature is defined as one below which thermally-induced denaturation or aggregation occurs at atmospheric conditions.

“High pressure” or “high hydrostatic pressure,” for the purposes of the invention is defined as pressures of from about 500 bar to about 40,000 bar. In some embodiments, the increased hydrostatic pressure may be from about 500 bar to about 10,000 bar, from about 500 bar to about 5000 bar, from about 500 bar to about 4000 bar, from about 500 bar to about 2000 bar, from about 500 bar to about 2500 bar, from about 500 bar to about 3000 bar, from about 500 bar to about 6000 bar, from about 1000 bar to about 5000 bar, from about 1000 bar to about 4000 bar, from about 1000 bar to about 2000 bar, from about 1000 bar to about 2500 bar, from about 1000 bar to about 3000 bar, from about 1000 bar to about 6000 bar, from about 1500 bar to about 5000 bar, from about 1500 bar to about 3000 bar, from about 1500 bar to about 4000 bar, from about 1500 bar to about 2000 bar, from about 2000 bar to about 5000 bar, from about 2000 bar to about 4000 bar, from about 2000 bar to about 3000 bar, or about 1000 bar, about 1500 bar, about 2000 bar, about 2500 bar, about 3000 bar, about 3500 bar, about 4000 bar, about 5000 bar, about 6000 bar, about 7000 bar, about 8000 bar, about 9000 bar.

Reduction of pressure: Where the reduction in pressure is performed in a continuous manner, the rate of pressure reduction can be constant or can be increased or decreased during the period in which the pressure is reduced. In some variations, the rate of pressure reduction is from about 5000 bar/1 sec to about 5000 bar/4 days (or about 3 days, about 2 days, about 1 day). Thus in some variations the rate of pressure reduction can be performed at a rate of from about 5000 bar/1 sec to about 5000 bar/80 hours, from about 5000 bar/1 sec to about 5000 bar/72 hours, from about 5000 bar/1 sec to about 5000 bar/60 hours, from about 5000 bar/1 sec to about 5000 bar/50 hours, from about 5000 bar/1 sec to about 5000 bar/48 hours, from about 5000 bar/1 sec to about 5000 bar/32 hours, from about 5000 bar/1 sec to about 5000 bar/24 hours, from about 5000 bar/1 sec to about 5000 bar/20 hours, from about 5000 bar/1 sec to about 5000 bar/18 hours, from about 5000 bar/1 sec to about 5000 bar/16 hours, from about 5000 bar/1 sec to about 5000 bar/12 hours, from about 5000 bar/1 sec to about 5000 bar/8 hours, from about 5000 bar/1 sec to about 5000 bar/4 hours, from about 5000 bar/1 sec to about 5000 bar/2 hours, from about 5000 bar/I sec to about 5000 bar/l hour, from about 5000 bar/1 sec to about 1000 bar/min, about 5000 bar/1 sec to about 500 bar/min, about 5000 bar/1 sec to about 300 bar/min, about 5000 bar/1 sec to about 250 bar/min, about 5000 bar/1 sec to about 200 bar/min, about 5000 bar/1 sec to about 150 bar/min, about 5000 bar/1 sec to about 100, about 5000 bar/1 sec to about 80 bar/min, about 5000 bar/1 sec to about 50 bar/min, or about 5000 bar/1 sec to about 10 bar/min. For example, about 10 bar/min, about 250 bar/S minute, about 500 bar/5 minutes, about 1000 bar/5 minutes, about 250 bar/5 minutes, 2000 bar/50 hours, 3000 bar/50 hours, 40000 bar/50 hours, etc. In some embodiments, the pressure reduction may be approximately instantaneous, as in where pressure is released by simply opening the device in which the sample is contained and immediately releasing the pressure.

Where the reduction in pressure is performed in a stepwise manner, the process comprises dropping the pressure from the highest pressure used to at least a secondary level that is intermediate between the highest level and atmospheric pressure. The goal is to provide an incubation or hold period at or about this intermediate pressure zone that permits a protein to adopt a desired conformation.

In some embodiments, where there are at least two stepwise pressure reductions there may be a hold period at a constant pressure between intervening steps. The hold period may be from about 10 minutes to about 50 hours (or longer, depending on the nature of the protein of interest). In some embodiments, the hold period may be from about 2 to about 30 hours, from about 2 to about 24 hours, from about 2 to about 18 hours, from about 1 to about 10 hours, from about 1 to about 8 hours, from about 1 to about 6 hours, from about 2 to about 10 hours, from about 2 to about 8 hours, from about 2 to about 6 hours, or about 2 hours, about 6 hours, about 10 hours, about 20 hours, or about 30 hours, from about 2 to about 10 hours, from about 2 to about 8 hours, from about 2 to about 6 hours.

In some variations, the pressure reduction includes at least 2 stepwise reductions of pressure (e.g., highest pressure reduced to a second pressure reduced atmospheric pressure would be two stepwise reductions). In other embodiments the pressure reduction includes more than 2 stepwise pressure reductions (e.g., 3, 4, 5, 6, etc.). In some embodiments, there is at least 1 hold period. In certain embodiments there is more than one hold period (e.g., at least 2, at least 3, at least 4, at least 5 hold periods).

In some variations of the methods the constant pressure after an initial stepwise reduction may be at a hydrostatic pressure of from about 500 bar to about 5000 bar, from about 500 bar to about 4000 bar, from about 500 bar to about 2000 bar, from about 1000 bar to about 4000 bar, from about 1000 bar to about 3000 bar, from about 1000 bar to about 2000 bar, from about 1500 bar to about 4000 bar, from about 1500 bar to about 3000 bar, from about 2000 bar to about 4000 bar, or from about 2000 bar to about 3000 bar.

In particular variations, constant pressure after the stepwise reduction is from about four-fifths of the pressure immediately prior to the stepwise pressure reduction to about one-tenth of prior to the stepwise pressure reduction. For example, constant pressure is at a pressure of from about four-fifths to about one-fifth, from about two-thirds to about one-tenth, from about two-thirds to about one-fifth, from about two-thirds to about one-third, about one-half, or about one-quarter of the pressure immediately prior to the stepwise pressure reduction. Where there is more than one stepwise pressure reduction step, the pressure referred to is the pressure immediately before the last pressure reduction (e.g., where 2000 bar is reduced to 1000 bar is reduced to 500 bar, the pressure of 500 bar is one-half of the pressure immediately preceding the previous reduction (1000 bar)).

Where the pressure is reduced in a stepwise manner, the rate of pressure reduction (e.g., the period of pressure reduction prior to and after the hold period) may be in the same range as that rate of pressure reduction described for continuous reduction (e.g., in a non-stepwise manner). In essence, stepwise pressure reduction is the reduction of pressure in a continuous manner to an intermediate constant pressure, followed by a hold period and then a further reduction of pressure in a continuous manner. The periods of continuous pressure reduction prior to and after each hold period may be the same continuous rate for each period of continuous pressure reduction or each period may have a different reduction rate. In some variations, there are two periods of continuous pressure reduction and a hold period. In certain embodiments, each continuous pressure reduction period has the same rate of pressure reduction. In other embodiments, each period has a different rate of pressure reduction. In particular embodiments, the hold period is from about 8 to about 24 hours. In some embodiments, the hold period is from about 12 to about 18 hours. In particular embodiments, the hold period is about 16 hours.

Combinations of the above conditions: Various combinations and permutations of the condition above, such as agitation of the protein under high pressure at an elevated temperature in the presence of chaotropes and redox reagents, can be employed as desired for optimization of refolding yields.

### High Pressure Devices and Considerations

Commercially available high pressure devices and reaction vessels, such as those described in the examples, may be used to achieve the hydrostatic pressures in accordance with the methods described herein (see BaroFold Inc., Boulder Co.). Additionally devices, vessels and other materials for carrying out the methods described herein, as well as guidance regarding the performing increased pressure methods, are described in detail in U.S. Pat. Nos. 6,489,450 7,064,192, which are incorporated herein in their entirety. The skilled artisan is particularly directed to column 9, lines 39-62 and Examples 2-4. International Pat. App. Pub. No. WO 02/062827, incorporated herein in its entirety, also provides the skilled artisan with detailed teachings regarding devices and use thereof for high hydrostatic pressure treatment of proteins throughout the specification. Particular devices and teachings regarding the use of high pressure devices is also provided in International Patent Application Publication No. WO 2007/062174, which is hereby incorporated by reference in its entirety.

Multiple-well sample holders may be used and can be conveniently sealed using self-adhesive plastic covers. The containers, or the entire multiple-well sample holder, may then be placed in a pressure vessel, such as those commercially available from the Flow International Corp. or High Pressure Equipment Co. The remainder of the interior volume of the high-pressure vessel may than be filled with water or other pressure transmitting fluid.

Mechanically, there are two primary methods of high-pressure processing: batch and continuous. Batch processes simply involve filling a specified chamber, pressurizing the chamber for a period of time, and repressurizing the batch. In contrast, continuous processes constantly feed aggregates into a pressure chamber and soluble, refolded proteins move out of the pressure chamber. In both set ups, good temperature and pressure control is essential, as fluctuations in these parameters can cause inconsistencies in yields. Both temperature and pressure should be measured inside the pressure chamber and properly controlled.

There are many methods for handling batch samples depending upon the specific stability issues of each target protein. Samples can be loaded directly into a pressure chamber, in which case the aqueous solution and/or suspension would be used as the pressure medium.

Alternately, samples can be loaded into any variety of sealed, flexible containers, including those described herein. This allows for greater flexibility in the pressure medium, as well as the surfaces to which the mixture is exposed. Sample vessels could conceivably even act to protect the desired protein from chemical degradation (e.g., oxygen scavenging plastics are available).

With continuous processing, small volumes under pressure can be used to refold large volumes the sample mixture. In addition, using an appropriate filter on the outlet of a continuous process will selectively release soluble desired protein from the chamber while retaining both soluble and insoluble aggregates.

Pressurization is a process of increasing the pressure (usually from atmospheric or ambient pressure) to a higher pressure. Pressurization takes place over a predetermined period of time, ranging from 0. I second to 10 hours. Such times include I second, 2 seconds, 5 seconds, 10 seconds, 20 seconds, 1 minute, 2 minutes, 5 minutes, to minutes, 30 minutes, 60 minutes, 2 hours, 3 hours, 4 hours, and 5 hours.

Depressurization is a process of decreasing the pressure, from a high pressure, to a lower pressure (usually atmospheric or ambient pressure). Depressurization takes place over a predetermined period of time, ranging from 10 seconds to 10 hours, and may be interrupted at one or more points to permit optimal refolding at intermediate (but still increased 30 compared to ambient) pressure levels. The depressurization or interruptions may be 1 second, 2 seconds, 5 seconds, 10 seconds, 20 seconds, 1 minute, 2 minutes, 5 minutes, 10 minutes, 30 minutes, 60 minutes, 2 hours, 3 hours, 4 hours, and 5 hours.

Degassing is the removal of gases dissolved in solutions and is often advantageous in the practice of the methods described herein. Gas is much more soluble in liquids at high pressure as compared to atmospheric pressure and, consequently, any gas headspace in a sample will be driven into solution upon pressurization. The consequences are two-fold: the additional oxygen in solution may chemically degrade the protein product, and gas exiting solution upon repressurization may cause additional aggregation. Thus, samples may be prepared with degassed solutions and all headspace should be filled with liquid prior to pressurization.

### EXAMPLE 1

### CTLA-4-Ig Aggregate Reductions

High pressure treatment of preparations with high monomer content under conditions identical to treatment of preparations of the same protein with high aggregate content leads to very different results. Studies have been conducted with CTLA-4-Ig that demonstrate effective refolding yields of solutions comprising >90% aggregates at 2000 bar, pH 7, at high and low ionic strength, with final aggregate compositions of 7% and 6% respectively. Monomer treated at high pressure (2000 bar) at low ionic strength maintained its native structure and size (1% aggregate). However, monomer treated at high pressure resulted in pressure induced aggregation, having a final composition of 7% aggregate. Therefore, refolding a sample that contained less than 90% aggregate cannot simply mimic conditions that are effective for refolding a solution with >90% aggregate. These results have a direct implication on immunogenicity concerns, since solutions after downstream purification will contain both monomer and aggregate. Accordingly, refolding high-monomer content protein solutions must be approached carefully, as seen in this instance for CTLA-4-Ig. An aggregated solution comprising 14% aggregate refolded at 2000 bar at high ionic strength resulted in a final aggregate composition of 6.4%. These aggregate levels are typically sufficient to generate immunogenicity in patients and not typically allowed by pharmaceutical regulatory authorities. However, if the CTLA-4-Ig solution comprising 14% aggregate is refolded at 2000 bar at low ionic strength the final aggregate composition is <1%. These refolding steps are effective in significantly reducing the potential of the protein solution to provoke immunogenicity.

Studies were conducted to determine procedures to reduce levels of soluble aggregates in CTLA-4-Ig (Orencia®, abatacept) fusion protein preparations. The CTLA-4-Ig fusion protein consists of the non-membrane bound portion of the CTLA-4 molecule (a dimer with an apparent molecular weight of 25 kDa) linked to the Fc domain of an antibody. The protein is glycosylated and has an apparent molecular weight of 92 kDa as analyzed by SDS-PAGE and light scattering.

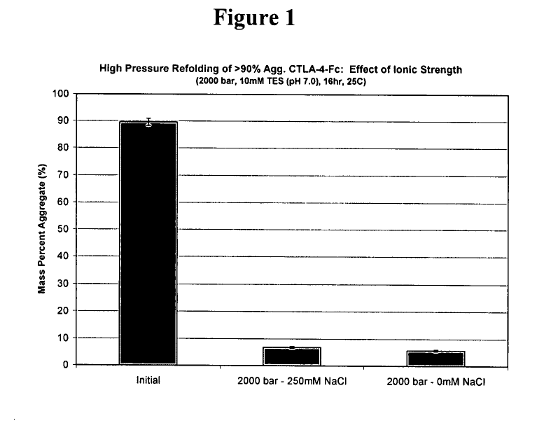

The studies were conducted on aggregated solutions of CTLA-4-Ig fusions containing 90+/−1% aggregate. Aggregated material was diluted to a protein concentration of 0.5 mg/ml in buffer solutions comprising of 10 mM TES (pH 7.0) containing either 0 or 250 mM NaCl. The aggregated solutions were pressure treated at 2000 bar for sixteen hours at 25C and analyzed for refolding. The results are shown in FIG. 1. After pressure treatment, the aggregate level decreased to 6.9%+/−0.4% and 5.8%+/−0.3%, as a function of ionic strength (250 mM and 0 mM NaCl respectively).

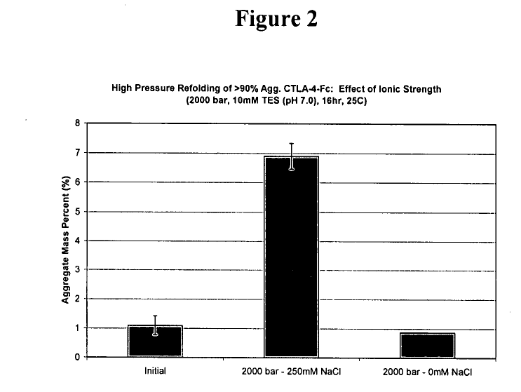

The stability of monomeric CTLA-4-Ig fusions was studied as a function of pressure and solution conditions. Monomeric CTLA-4-Ig was pressure treated in 10 mM TES (pH 7.0) as a function of ionic strength (0 and 250 mM NaCl) at a protein concentration of 0.5 mg/ml. Pressure was found to induce aggregation in solutions containing salt resulting in a final aggregate concentration of approximately 7% (FIG. 2). The protein remained aggregate free after pressure treatment in conditions that did not contain salt.

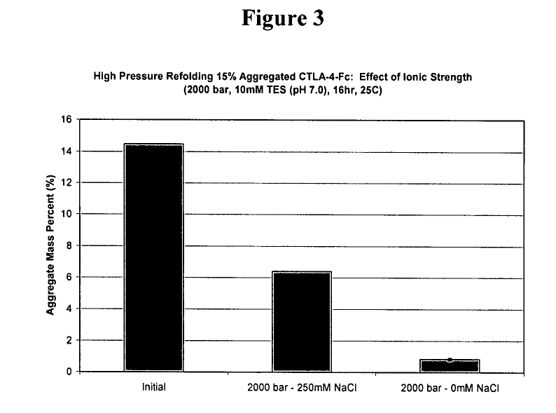

High pressure studies were conducted to refold aggregated solution of CTLA-4-Ig fusions containing 14.5+/−0.1% aggregated (“moderate” aggregate levels). Aggregated material was diluted to a protein concentration of 0.5 mg/ml in buffer solutions of 10 mM TES containing either 0 or 250 mM NaCl. The aggregated solutions were pressure treated at 2000 bar for sixteen hours at 25C and analyzed for refolding. High pressure treatment resulted in a reduction of aggregate levels in the buffer containing 250 mM NaCl, with a final percentage (6%) that would typically be excessive for a pharmaceutical product. At lower ionic strengths, the aggregate level was reduced to less than 1%, essentially eliminating aggregates from the sample. The results are shown in FIG. 3. After pressure treatment, the aggregate level decreased to 6.9%+/−0.4% and 5.8%+/−0.3%, as a function of ionic strength (250 mM and 0 mM NaCl respectively).

In order to mimic solutions of protein aggregates that could result from initial rounds of purification, CTLA-4-Ig solutions comprising moderate levels of aggregate (˜18%) were prepared. Commercial formulations of CTLA-4-Ig fusions were diluted to a protein concentration of 12 mg/ml and incubated at pH 3 for 3 hours at 23° C. to induce aggregation. Two runs resulted in final aggregate concentrations of 15% and 21% respectively. Aggregate analysis was quantified by SE-HPLC. Highly-aggregated (>90%) preparations of CTLA-4-Ig were also made. CTLA-4-Ig aggregates were prepared by dissolving 66 mg of lyophilized cake in 1 ml of water. The vial was silicon oil free. This stock was diluted to final protein concentration of 5 mg/ml in buffer containing 10 mM Citrate, pH 3, 240 mM NaCl. This was allowed to sit at ambient conditions for 17 days which produced ˜85% aggregate. On day 19 the solution was rapidly shaken to induce further aggregation for 1 hour at ambient conditions. Visible precipitate was observed at this point. The solution was immediately used in refolding. SEC analysis of the remaining soluble material was used to establish >90% aggregate levels.

For pressurization, pressure was increased at a rate of 500 bar/minute until the desired pressure was achieved. During refolding, the temperature was maintained at 22° C. (R.T.). The samples were held under pressure for approximately 16 hours and then were depressurized at a rate of 250 bar/five minutes. The samples were immediately prepared for SE-HPLC after depressurization.

SE-HPLC analysis of protein fractions was conducted on a Beckman Gold HPLC system (Beckman Coulter, Fullerton, Calif.) equipped with a TSK G3000 SW_{XL} size exclusion column (Tosohaas). A filtered mobile phase of PBS (pH 7.2) at a rate of 1.0 ml/min was used, with an 10-25 ug protein sample injection from a Beckman 507e autosampler. Absorbance was monitored at 215 nm.

### EXAMPLE 2

### Recombinant Human Growth Hormone (rhGH) Refolding Studies

Studies were undertaken to determine “best case” conditions for refolding of recombinant human growth hormone (rhGH) for use in subsequent studies on the immunogenicity of rhGH (see the Example entitled “Recombinant human growth hormone (rhGH) refolding studies” below). Investigations by St. John et al. have demonstrated that rhGH is sensitive to aggregation by shaking (St. John, R. J., J. F. Carpenter, et al. (2001), Journal of Biological Chemistry 276(50): 46856-46863). Two types of aggregates were generated by gentle agitation in formulation buffer or formulation buffer containing 0.75M guanidine HCl (ibid). Formulation buffer was defined as 10 mM Na Citrate (pH 6.0), 1 mM EDTA, 0.1% sodium azide and the resulting aggregated rhGH solutions were found to contain >90% aggregates (ibid). High pressure refolding studies were conducted to determine the effect of pressure, temperature, and guanidine HCl concentration on the refolding of the two types of shake-induced aggregates. For aggregates formed in formulation buffer alone, high pressure treatment at 2000 bar for 48 hrs at a protein concentration of 0.87 mg/ml resulted in the a greater than 90% recovery of soluble rhGH (ibid). Likewise, aggregates formed in formulation buffer containing 0.75M guanidine refolded with >90% yields by pressure treatment at 2000 bar with the addition of 1 M guanidine at identical pressure, protein concentration, and incubation times (ibid). These conditions were adopted as “best case” conditions for refolding of rhGH. If guanidine was not added to the refolding mixture, refolding yields did not exceed 20% (ibid). Elevated temperatures were also found to play a significant role in refolding these aggregates. Refolding of both types of aggregates at temperatures of 2000 bar in refolding buffer at a protein concentration of 0.65 mg/ml resulted in at least 90% recovery of rhGH in a soluble form (ibid). If the identical refold was conducted at 25° C., recoveries of less than 20% were obtained (ibid).

Somatropin® (rhGH) was purchased commercially in its liquid formulation. 200 ul of the material (at a concentration of 10 mg/ml) was dialyzed overnight against buffer containing 10 mM Na Citrate (pH 6.0) using Pierce® microdialysis cups. This material was then placed in its final pressure treatment condition by the appropriate addition of EDTA from a 500 mM stock and guanidine from a 6M stock. Pressure treatment was conducted as described by St. John, R. J., J. F. Carpenter, et al. (2001), Journal of Biological Chemistry 276(50): 46856-46863.

A Superdex 75 10/300 GL column was used for the SE-HPLC assay. A Beckman Coulter System Gold HPLC with 126 solvent module and Waters autosampler were used online with an ultraviolet detector set at a wavelength of 280 nm. The mobile phase buffer was Phosphate Buffered Saline at a flowrate of 0.6 ml/min and a sample injection of 50 μl. The samples were kept at 4° C. in the autosampler until injection. Data was collected over a period of 90 minutes.

The stability of monomeric rhGH as a function of guanidine HCl concentration and temperature and pressure was examined in buffer containing 10 mM Na Citrate (pH 6.0), 1 mM EDTA, 0.1% sodium azide. Monomeric Somotropin® was dialyzed into formulation buffer and pressure treated at 2000 bar at 60° C. and at 2000 bar with the addition of 0.25, 0.5, 0.75, 1, 1.5, and 2M guanidine HCl at 25° C. None of the treatments induced aggregation of monomeric rhGH as determined by SE-HPLC. It was thus hypothesized that these conditions would be effective for the refolding of solutions containing moderate amounts of aggregates and would be effective for reducing the immunogenicity of rhGH formulations that contained aggregates. However, these results demonstrate that there are additional contstraints on the refolding process for reducing immunogenicity for downstream processing applications since )1 elevated temperatures accelerate chemical degradation pathways that lead to non-homogenous pharmaceutical products (Manning et al., Pharmaceutical Research, v6, 903-918, 1989) and 2) reagents added to the refolding process must be easily removed prior to final formulation In this case, guanidine HCl cannot be present in the final formulation due to its toxicity. Consequently, further consideration of the techniques used for refolding must be considered. This example illustrates the need to examine monomer stability during pressure treatment.

### EXAMPLE 3

### Recombinant Human Growth Hormone (rhGH) Immunogenicity Studies

Studies were conducted to determine the effect of aggregates before and after high pressure treatment on the immunogenicity response in naïve mice dosed with varying forms of rhGH, in a similar immunogenicity model taught by Braun et al. (Braun et al., Pharmaceutical Research, v14, pg. 1472-1478, 1997).

rhGH samples were produced from Nordiflex (a liquid formulation of rhGH manufactured by NovoNordisk). 15 mg vials of Nordiflex were purchased from the University of Colorado apothecary. The rhGH was diluted to a concentration of 1 mg/ml. The diluent used was one of two conditions: (1) the formulation buffer or (2) formulation buffer without pluronic F-68. The Norditropin formulation buffer contains 1.7 mg histidine, 4.5 mg pluronic F-68, phenol 4.5 mg, mannitol 58 mg in 1.5 ml of water as a diluent. The diluted samples were then either shaken (described as “Shaken”) or stressed using freeze-thaw cycles (described as “FT”) to investigate the formation of aggregates.

Freeze-thaw samples were made by diluting Nordiflex in the appropriate formulation buffer. A volume of 0.75 ml rhGH at a protein concentration of 1 mg/ml Nordiflex was inserted in a 2 ml polypropylene tube and placed into liquid nitrogen for one minute to ensure complete freezing. The samples were then placed in 22° C. water and allowed to thaw for ten minutes. The cycle was repeated for a total of 20 cycles.

For a monomeric control, an additional sample of untreated Nordiflex was diluted in formulation buffer (1 mg/ml Nordiflex) and not subjected to any stressful conditions.

The effect of pressure on this material was examined by splitting the samples and placing the samples (freeze-thaw 20×, and monomeric control) at a pressure of 2000 bar at 70° C. overnight.

For pressurization, pressure was increased at a rate of 500 bar/minute until a pressure of 2000 bar was achieved. At 2000 bar, the temperature of the high pressure vessel was increased to 70° C. and the samples incubated for 16 hours. Prior to depressurization (250 bar/5 min), the pressure vessel was cooled to room temperature. The samples were immediately prepared for SE-HPLC after depressurization.

A Superdex 75 10/300 GL column was used for the Size-Exclusion Chromatography (SE-HPLC) assay. A Beckman Coulter System Gold HPLC with 126 solvent module and Waters autosampler were used online with an ultraviolet detector set at a wavelength of 280 nm. The mobile phase buffer was Phosphate Buffered Saline with a flow rate of 0.6 ml/min. The sample injection size was 50 μl. The samples were kept at 4° C. in the autosampler until injection. Data was collected over a period of 90 minutes.

6 week old naïve mice were dosed with 10 ug of monomeric rhGH and 10 ug of “FT” aggregates of rhGH, and 10 ug of “FT” aggregates treated with high pressure, described as “HP FT Aggregates”. Dosing was conducted on days 7, 14, and 21. Buffer was also dosed at identical volumes and times as a control.

Prior to collecting blood, the mice were anesthetized using isofluorane inhalant gas. Each mouse was held, singly, with its nose in a tube of steady flow of isofluorane inhalant gas. Once the mouse had taken at least 10 deep breaths and gone limp, the flow was reduced from 5% to 3-4%. A drop of Proparacaine was applied to a single eye after the mouse was no longer responsive to a toe pinch. Blood was then collected from the retro-orbital venous sinus twice using 50 μl capillary tubes. The mouse continued to be sedated with the isofluorane inhalant gas throughout the blood collection process. After sufficient blood was collected, ˜100 μl, the eye was blotted with sterile gauze and an additional drop of Proparacaine was administered. Gentle pressure was used to hold the affected eye shut for 1-2 minutes. Next, the mouse was injected intraperitoneally with a 100 μl injection containing 10 μg of human growth hormone in an isotonic, buffered solution that has been subjected to one of four conditions (i.e., (1) vigorous shaking (2) freeze-thaw (3) high-pressure treated (4) suggested manufacturers storing conditions). The mice were labeled using ear punches. Each mouse received 10 μg of protein in a single 0.1 ml injection. This dose interval and amount was determined from previous work (Hermeling, S., W. Jiskoot, et al. (2005), Pharmaceutical Research 22(6): 847-851). Bleeds conducted on days 0, 7, 14, 21, and 28 with eight female mice in each group.

The sera collected were tested for specific antibody response through the use of ELISA. The wells of Immulon 4 High Binding Affinity (HBA) plates were incubated with 200 μl of a diluted rhGH (16 μg/ml) prepared from the Norditropin formulation at lab temperature overnight with gentle agitation. The wells were then drained and washed three times with 1× Phosphate Buffered Saline (PBS). After the final wash the wells were tapped dry on a paper towel. The wells were then blocked with 200 μl of a 1× PBS, 1% Bovine Serum Albumin (BSA) solution for 1 hour. Upon adsorption of the blocking solution the wells were washed three times with a solution of 1×PBS. Wells in rows B-H were then loaded with 100 μl of dilution buffer (200 mM HEPES, 50 mM disodium EDTA, 750 mM sodium chloride with 1% BSA and 0.1% triton ×100). The sera were then diluted 1:20 into the dilution buffer and added to the wells in row A. Using a multichannel pipet, 100 μl of the sera dilutions from row A were transferred to the wells in row B (1:2 dilution). The solution in row B is mixed by drawing up and expelling 100 μl 5 times into the wells before transferring 100 μl to wells in row C. The 2× dilutions were continued through row G. The plates were then sealed and allowed to incubate at lab temperature for 30 minutes. The wells were then washed three times with a solution of 200 mM HEPES, 50 mM disodium EDTA, 750 mM sodium chloride and 0.1% triton X-100 and tapped dry on a paper towel. The wells were then incubated with 100 μl of a horse radish peroxidase (HRP) conjugated goat anti-mouse IgG (Chemicon) diluted 1:8000 into dilution buffer. After 1 hour the wells were washed three times with 1×PBS and tapped dry on a paper towel followed by the addition of 100 μl of 3,3′,5,5′ tetramethylbenzidine (TMB) to each well. After 20 minutes 50 μl of 0.5 M sulfuric acid was added to the wells to quench the reaction. The absorbance was recorded with a Molecular Devices “V max” kinetic plate reader at a wavelength of 450 nm and a reference wavelength of 595 nm. The ELISA response is reported as a concentration of binding antibody present as calculated by comparing the absorbance to the linear portion of a standard curve and multiplied by the dilution factor.

The data was modeled as a general factorial design with 1 response and levels appropriate to the number of groups in each study. Each group had eight replicates. The software program Stat-Ease 7.2.1 was used to conduct a linear analysis of variance (ANOVA). The probability of a [t] between means of groups was compared with a 90% confidence interval. When comparing means, probabilities of [t]<0.1 were significant based on the 90% confidence interval chosen.

FT aggregates of Nordiflex were found to contain 77% aggregates, with 85% of the aggregate being insoluble. After high pressure treatment, the aggregate level was reduced to 5%.

Six week old naïve mice were dosed with 10 ug of monomeric rhGH, 10 ug of “FT” aggregates of rhGH, and 10 ug of aggregates treated with high pressure, described as “HP FT Aggregates”. All samples were generated using Nordiflex as a starting material. Dosing was conducted on days 7, 14, and 21 with bleeds conducted on days 0, 7, 14, 21, and 28 with eight female mice in each group. Buffer was also dosed at identical volumes and times as a control.

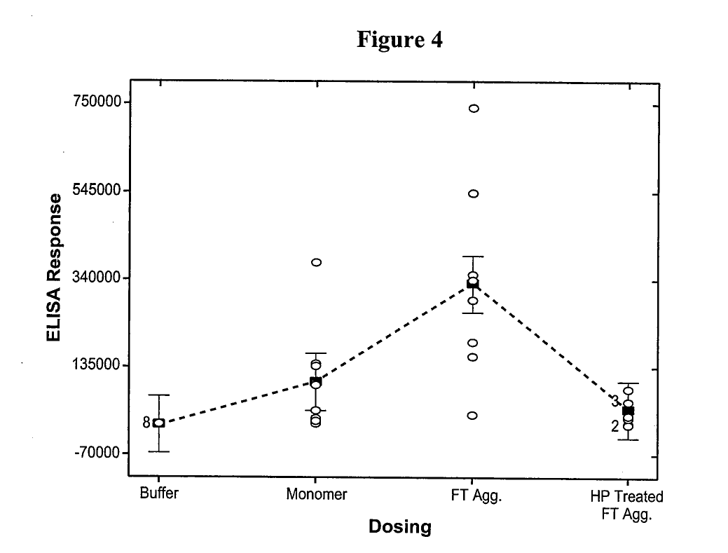

ELISAs were conducted on the bleeds to detect the presence of antibodies against monomeric growth hormone. The fourth bleed provided the highest ELISA response (data not shown). A box plot of the ELISA response in the fourth bleed is shown in FIG. 4. The results of the study demonstrate that “FT” aggregates of rhGH generated a significant response (Probability >[t] of <0.0001) relative to high pressure treated FT aggregates. Monomeric rhGH generated a subtle immune response relative to buffer (Probability >[t] of 0.07), which is expected considering the natural immunogenicity of human proteins in mice. “HP FT Aggregates” generated an immune response in mice that was not significantly different [Probability >[t] of 0.245] to mice dosed with monomeric rhGH, demonstrating the monomeric nature and reduced immunogenicity of high pressure treated aggregates. Statistical analysis was conducted to determine that only the 21 day bleed contained an antibody response that was significant over baseline.

### EXAMPLE 4

### Human Interferon-Beta-1b (IFN-Beta) Studies

Refolding conditions for material which is highly aggregated are not necessarily useful for refolding highly-monomeric material, as the following example demonstrates. Human interferon-beta-1b (IFN-beta) is a therapeutic protein used for the treatment of multiple sclerosis. The original process for the expression, refolding and production of IFN-beta is described in U.S. Pat. No. 4,462,940 (Hanisch and Fernandes 1983; Konrad and Lin 1984). The wild type protein has been mutated at the C17 site to remove a free cysteine and thus has only 1 disulfide and a molecular weight of ˜20 kDa. The pI of the protein is 8.9.

A variation of the method taught by Shaked et al. was used to produce IFN-beta (U.S. Pat. No. 5,183,746). IFN-beta was purified from inclusion bodies by extraction by sec-butanol. Following acid precipitation, the material was purified using one SE-HPLC column operated in SDS, in contrast to the two column steps used in the Shaked method. Oxidation of IFN-beta occurred in a method similar to the method taught previously. After oxidation, the material was buffer exchanged into a solution containing 0.1% sodium laurate, pH 9.0. This step was used to remove any SDS bound to the protein. The sodium laurate was precipitated by adjusting the pH to 3.0. Aggregates of IFN-beta were then separated from monomeric forms by a second SE-HPLC method, using a Tosoh Biosciences 2000SW_{XL}. Aggregates comprised 30-40% of the purification step.

SE-HPLC analysis of protein fractions was conducted on a Beckman Gold HPLC system (Beckman Coulter, Fullerton, Calif.) equipped with a TSK G2000 SW_{XL} size exclusion column (Tosohaas). A filtered mobile phase of 10 mM HCl (˜pH 2.0) at a rate of 0.5 of 1 ml/min was used, with an 10-25 ug protein sample injection from a Beckman 507e autosampler. Absorbance was monitored at 215 nm.

When purified monomer of IFN-beta was treated under refolding conditions useful for the refolding of inclusion bodies (solution comprising >90% aggregates), the high pressure treatment resulted in aggregation, increasing the aggregate content from 0.1% to 29+/−2% as determined by SE-HPLC.

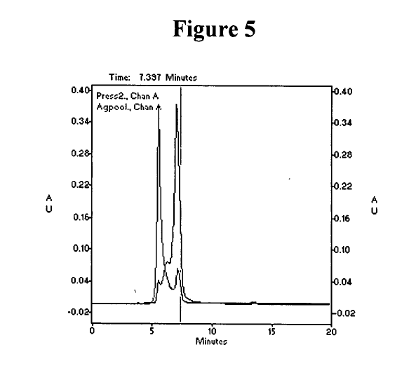

IFN-beta aggregates (solution comprising 80% aggregates) were formed following the SDS refolding and purification process taught in U.S. Pat. Nos. 4,462,940 and 5,183,746. After sodium laurate precipitation, the monomer and aggregate fractions were separated by sizing in using 10 mM HCl running buffer and formulated in buffer containing 10 mM HCl. The aggregate fractions were pressure treated at 2700 bar at 25C for 16 hrs at a protein concentration of 80 ug/ml. Depressurization at 250 bar/min was used. As shown in FIG. 5, high pressure treatment resulted in a majority of the aggregate being converted from aggregate (left peak) to monomer (right peak) in solution conditions that would not be applicable for the refolding of inclusion bodies. This example demonstrates that refolding conditions for reducing immunogenicity after process purification are not useful for refolding inclusion bodies.

### EXAMPLE 5

### Recombinant Murine Interferon-Beta (rmIFN-beta)

Studies were conducted to determine the effect of aggregates before and after high pressure treatment on the immunogenicity response in mice dosed with varying forms of rmIFN-beta. In contrast to the rhGH hormone study described previously, there should be no inherent immune reaction to the dosed protein since it is of murine origin.

To prepare monomeric rmIFN-beta, monomeric rmIFN-beta was purchased from PBL Biomedical laboratories and dialyzed into buffer containing 20 mM histidine (pH 6.0), 166 mM NaCl, and 6% glycerol. The dialysis step induced aggregation, however the aggregates could be removed by centrifugation. The soluble fraction was analyzed by SE-HPLC and was found to be entirely monomeric. The higher glycerol content in this sample occurred due to the unexpected loss of protein during the dialysis step. Consequently, this sample was not diluted as anticipated. The material was sterile filtered prior to dosing and SE-HPLC analysis was conducted to ensure that filtration did not induce aggregation.

To generate aggregated rmIFN-beta, monomeric rmIFN-beta (0.33 mg/ml) was purchased from PBL Biomedical laboratories, sterile filtered, and aggregated by agitation at a vortex level of 3 for 5 minutes. The material was diluted 1:3 to generate final material that contained 53% insoluble aggregate, 7% soluble aggregate, and 40% monomer at a protein concentration of 0.1 mg/ml. Aggregate content was determined by SE-HPLC. The material was formulated in a buffer containing 20 mM histidine (pH 6.0), 166 mM NaCl, 2% glycerol.

Insoluble aggregates of rmIFN-beta (see generation of aggregated material) were resuspended in refolding buffer containing 20 mM histidine (pH 6.0), 166 mM NaCl, 2% glycerol and pressure treated at 2000 bar for 16 hours at 25° C. Depressurization was conducted at a rate of 250 bar/5 minutes. The pressure-modulated refolding yield was calculated to be 39% by SE-HPLC, however the insoluble material was removed by centrifugation to generate material that was aggregate free (SE-HPLC) after sterile filtration.

C57B1/6 mice (6-7 week old, female) were dosed with monomeric (100% monomer), aggregated (53% insoluble aggregates, 7% soluble aggregate, 40% monomer) or high pressure treated aggregates (100% monomer) at dosing levels of 0.5 and 2.3 ug/day on days 1-5, 8-12, and 15-20, as described below. Orbital bleeds were taken on days 8, 15, 23, with the terminal bleed occurring on day 40. The development of antibodies to monomeric IFN-beta as a function of the different doses was monitored using an internally developed ELISA.

C57B1/6 mice (6-7 week old, female) were dosed at Washington Bio with monomeric (100% monomer), aggregated (53% insoluble aggregates, 7% soluble aggregate, 40% monomer) or high pressure treated aggregates (100% monomer) at dosing levels of 0.5 and 2.3 ug/day. There were eight mice per group, dosed on days 1-5, 8-12, and 15-20 with orbital bleeds taken on days 8, 15, 23, and the terminal bleed occurring on day 40 per protocol PK-BF-1. Blood samples were aliquoted in two vials and stored at −70° C. prior to shipment to BaroFold on dry ice. Samples were kept at −70° C. prior to analysis via ELISA.

The sera collected were tested for specific antibody response through the use of ELISA. The wells of Immulon 4 High Binding Affinity (HBA) plates were coated with 150 μl of a diluted rMuIFN-0 (250 ng/ml in 50 mM carbonate-bicarbonate buffer pH 9.5) prepared from the rMuIFN-β (PBL Biomedical Laboratories) at lab temperature overnight. The wells were then drained and washed two times with 1× Phosphate Buffered Saline (PBS). After the final wash the wells were tapped dry on a paper towel. The wells were then blocked with 200 μl of a 1% Bovine Serum Albumin (BSA) in 40 mM HEPES, 10 mM EDTA, 150 mM NaCl pH 7.4 solution for 1 hour. Upon adsorption of the blocking solution the wells were washed three times with a solution of 1× PBS. Plates not being used immediately were then coated with 200 μl of 10% Sucrose and allowed to stand for 10 minutes. Sucrose solution was drained from the wells and plates were sealed and stored at 4° C. until needed. Studies were conducted to ensure that there was no loss of efficacy of plates stored for 1 week.

Plates were equilibrated by loading 150 μl of dilution buffer (1% BSA, 0.1% Triton X-100, 40 mM HEPES, 10 mM EDTA, 150 mM NaCl pH 7.4) into each well and allowed to incubate at room temperature for 20 minutes before removing residual material. Wells in rows B-H were then loaded with 100 μl of dilution buffer. Wells A1 and A2 were loaded with 150 μl of standard monoclonal antibody (rat anti-MuIFN-β from PBL Biomedical Laboratories)(200 ng/ml) The sera were then diluted 1:10 into the dilution buffer and 150 μl added to the wells in row A. The samples were then diluted 1:3 down the ELISA, with the last row serving as a blank. The plates were then sealed and allowed to incubate at lab temperature for 60 minutes. The wells were then washed three times with a solution of wash buffer 1 (40 mM HEPES, 10 mM EDTA, 150 mM NaCl pH 7.4 and 0.1% triton X-100) and tapped dry on a paper towel.

The wells containing standard were then incubated with 100 μl of a horse radish peroxidase (HRP) conjugated goat anti-rat IgG (Chemicon) diluted 1:15,000 into dilution buffer. The wells containing samples were incubated with 100 μl of a horse radish peroxidase (HRP) conjugated goat anti-mouse IgG (Chemicon) diluted 1:2000 into dilution buffer. After 1 hour the wells were washed two times with wash buffer 1 and once with 1×PBS and tapped dry on a paper towel. Each well was loaded with 100 μl of 3,3′,5,5′ tetramethylbenzidine (TMB) to each well. After 20 minutes 50 μl of 0.5 M sulfuric acid was added to the wells to quench the reaction. The absorbance was recorded with a Molecular Devices “V max” kinetic plate reader at a wavelength of 450 nm and a reference wavelength of 595 nm.

ELISA responses are reported as the Absorbance reading at 450 nm, multiplied by a dilution factor on the linear portion of the standard curve. Examination was conducted to ensure that the selection of the dilution factor did not affect the results.

SE-HPLC analysis of protein fractions was conducted on a Beckman Gold HPLC system (Beckman Coulter, Fullerton, Calif.) equipped with a TSK G2000 SW_{XL} size exclusion column (Tosohaas). A filtered mobile phase of 10 mM HCl (˜pH 2.0) at a rate of 0.5 of 1 ml/min was used, with an 10-25 ug protein sample injection from a Beckman 507e autosampler. Absorbance was monitored at 215 nm.

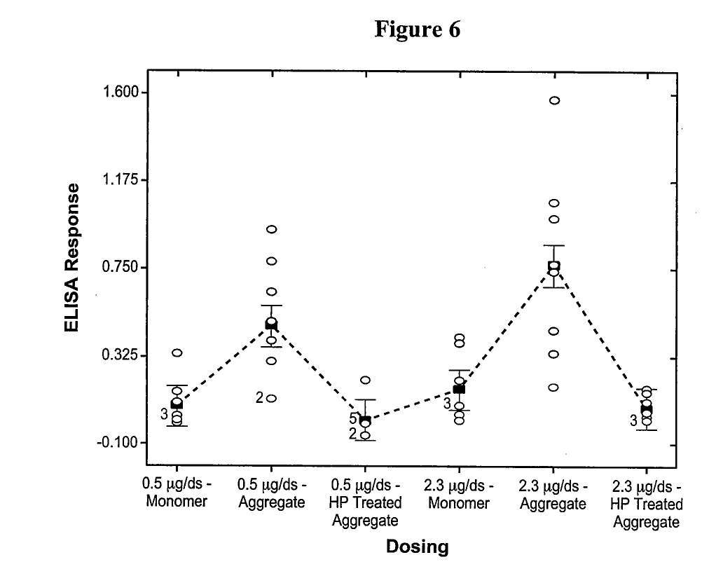

Data analysis of the Day 23 bleed (conducted after dosing was completed) demonstrates that only mice dosed with aggregates of rmIFN-beta had a significant immune response relative to the monomeric control Probability >[t] less than 0.0001. Additionally, higher dosing of the aggregate resulted in an increased response Probability >[t] of 0.019 and was not mirrored in either the animals that were dosed with monomeric IFN-beta or dosed with high pressure treated aggregates. Animals dosed with either monomer or HP treated aggregates had immune responses that were not significantly different. Analysis of the eight day bleed demonstrated a baseline response, demonstrating the positive response in animals subjected to fifteen days of aggregate dosing (see FIG. 6, ELISA response of naïve mice dosed with monomer, aggregated, and high pressure treated aggregates of rmIFN-beta—dosing was conducted at either 0.5 ug/dose or 2.3 ug/dose for fifteen days).

What is claimed is:

1. A high pressure treated therapeutic protein composition having reduced immunogenicity, comprising an isolated protein and a pharmaceutically acceptable carrier.

2. The therapeutic protein composition of claim 1, wherein the immune response of an individual to the therapeutic protein composition is reduced by at least about 50% as compared to the immune response to a composition of the same protein having no treatment by high pressure.

3. The therapeutic protein composition of claim 2, wherein the protein is endogenous to the species of the individual.

4. The protein composition of claim 1, wherein the protein composition contains less than about 10% of aggregated protein as a percentage of total protein after to high pressure treatment.

5. The protein composition of claim 1, wherein the protein composition contains less than about 5% of aggregated protein as a percentage of total protein after to high pressure treatment.

6. The protein composition of claim 1, wherein the protein composition contains less than about 1% of aggregated protein as a percentage of total protein after to high pressure treatment.

7. The protein composition of claim 5, wherein the amount of aggregated protein is measured by a method selected from the group consisting of analytical ultracentrifugation, size exclusion chromatography, field flow fractionation, light scattering, light obscuration, fluorescence spectroscopy, gel electrophoresis, GEMMA analysis, and nuclear magnetic resonance spectroscopy.

8. A protein composition, comprising an isolated protein and a pharmaceutically acceptable carrier, where the immune response to the therapeutic protein composition treated by high pressure is reduced by at least about 50% as compared to the immune response to the composition of the same protein prior to treatment by high pressure in a transgenic animal carrying a transgene encoding the protein.

9. A protein composition, comprising an isolated protein and a pharmaceutically acceptable carrier, where the immune response to the therapeutic protein composition treated by high pressure is reduced by at least about 50% as compared to the immune response to the composition of the same protein prior to treatment by high pressure in an animal with induced tolerance to the protein.

10. The protein composition of claim 9, wherein tolerance is induced by neonatal exposure to the protein.

11. The composition of claim 1, wherein the protein composition treated by high pressure has a soluble aggregate concentration at least about 50% lower than the protein composition prior to treatment with high pressure.

12. A method of preparing a therapeutic protein preparation comprising the composition of claim 1 for administration, comprising:
a) subjecting the therapeutic protein preparation to high pressure and solution conditions that do not induce aggregate formation;
b) releasing the pressure; and
c) administering the therapeutic protein preparation to an individual.

13. The method of claim 12, wherein the high pressure is between about 1000 bar and 3500 bar.

14. The method of claim 12, wherein the therapeutic protein preparation is administered to the individual within about 6 months of releasing the pressure.

15. The method of claim 12, wherein the high pressure or solution conditions include conditions selected from magnitude of high pressure, duration of high-pressure treatment, protein concentration, temperature, pH, ionic strength, chaotrope concentration, surfactant concentration, buffer concentration, and preferential excluding compound concentration.

16. The method of claim 12, where the immune response of the individual to the therapeutic protein composition treated by high pressure is reduced by at least about 50% as compared to the immune response of the individual to the composition of the same protein prior to treatment by high pressure.

17. The method of claim 12, wherein the therapeutic protein composition treated by high pressure has a soluble aggregate concentration at least about 50% lower than the therapeutic protein composition prior to treatment with high pressure.

18. A method of comparing the immunogenicity of a high-pressure treated protein to the same protein which has not been treated with high pressure, comprising:
a) subjecting a solution of the protein to high-pressure treatment;
b) before or after step a, placing the high-pressure treated protein in a pharmaceutically acceptable carrier if it is not already in such a carrier;
c) administering the high-pressure treated protein to a first individual;
d) at any point in the method, placing the non-high-pressure treated protein in a pharmaceutically acceptable carrier if it is not already in such a carrier;
e) at any point in the method after placing in a pharmaceutically acceptable carrier, administering the non-high-pressure treated protein to a second individual; and
f) comparing the immune response of the first individual to the second individual;
wherein a reduced immune response of the first individual as compared to the second individual indicates that the high-pressure treated protein has reduced immunogenicity.

19. The method of claim 18, wherein the immune response is assayed by antibody levels or antibody titers, a Biacore assay, or a clinical immune reaction.

20. The method of claim 18, wherein the first and second individuals are transgenic animals and the transgene expresses the protein used in the method.

21. The method of claim 18, wherein the first and second individuals are tolerized to the protein used in the method.

22. The method of claim 18, wherein the administering the high-pressure treated protein to a first individual takes place at least about 6 months after release of the high pressure.
